(19) United States
(12) Patent Application Publication
(10) Pub. No.: US 2003/0026780 A1
(43) Pub. Date: Feb. 6, 2003

(54) Innate immunity mediated methods of treating pathological conditions

(76) Inventors: Leroy E. Hood, Seattle, WA (US); Alan Aderem, Seattle, WA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/910,144

(22) Filed: Jul. 18, 2001

(51) Int. Cl. A61K 38/21; A61K 39/00

(52) U.S. Cl. 424/85.5; 424/185.1

(57) ABSTRACT

The invention provides a composition containing two or more ADCC targeting molecules, each selective for different antigens on the surface of a target cell, and in a pharmaceutically acceptable medium. The composition can contain more than two binding species. The composition also can be a pentameric binding molecule. Also provided is a composition containing effector cells and two or more ADCC targeting molecule species in a pharmaceutically acceptable medium. The invention further provides a method of inducing antibody-dependent cell cytotoxicity (ADCC) against a target cell. The method consists of contacting the target cell in the presence of effector cells with two or more ADCC targeting molecule species each selective for different antigens on the surface of the target cell. A method of treating a pathological condition characterized by aberrant cell growth is also provided. The method consists of administering an effective amount of two or more ADCC targeting molecules selective for different antigens expressed on the surface of cells mediating the pathological condition.

This application claims the benefit of U.S. Provisional Application No. 60/______, filed Jul. 18, 2000, which was converted from U.S. Ser. No. 09/618,176, and is incorporated herein by reference.

### BACKGROUND OF THE INVENTION

This invention relates generally to proliferative diseases such as cancer and, more specifically, to antibody-dependent cell cytotoxicity to treat and reduce the severity of proliferative diseases.

Cancer is currently the second leading cause of mortality in the United States. However, it is estimated that by the year 2000 cancer will surpass heart disease and become the leading cause of death in the United States. Prostate cancer in particular is the most common non-cutaneous cancer in the United States and the second leading cause of male cancer mortality. Cancerous tumors result when a cell escapes from its normal growth regulatory mechanisms and proliferates in an uncontrolled fashion. Tumor cells can metastasize to secondary sites if treatment of the primary tumor is either not complete or not initiated before substantial progression of the disease. Early diagnosis and effective treatment of tumors is therefore essential for survival.

Continuous developments over the past quarter century have resulted in substantial improvements in the ability of a physician to diagnose a cancer in a patient. Unfortunately, methods for treating cancer have not kept pace with those for diagnosing the disease. Thus, while the death rate from various cancers has decreased due to the ability of a physician to detect the disease at an earlier stage, the ability to treat patients presenting with more advanced disease has advanced only minimally.

A major hurdle to advances in treating cancer is the relative lack of agents that can selectively target the cancer, while sparing normal tissue. For example, radiation therapy and surgery, which generally are localized treatments, can cause substantial damage to normal tissue in the treatment field, resulting in scarring and, in severe cases, loss of function of the normal tissue. Chemotherapy, which generally is administered systemically, can cause substantial damage to organs such as bone marrow, mucosae, skin and the small intestine, which undergo rapid cell turnover and continuous cell division. As a result, undesirable side effects, for example, nausea, hair loss and reduced blood cell counts, occur as a result of systemically treating a cancer patient with chemotherapeutic agents. Such undesirable side effects often limit the amount of a treatment that can be administered. Moreover, cytotoxic agents have been combined with antibodies directed to the tumor cell surface markers. However, such approaches have suffered from the lack of availability or specificity of tumor cell markers. Due to such shortcomings in treatment, cancer remains a leading cause of patient morbidity and death.

Thus, there exists a need for improved methods of treating cancer and other proliferative pathological conditions. The present invention satisfies this need and provides related advantages as well.

### SUMMARY OF THE INVENTION

The invention provides a composition containing two or more ADCC targeting molecules in a pharmaceutically acceptable medium. The composition can contain more than two binding species. The composition also can be a pentameric binding molecule. Also provided is a composition containing effector cells and two or more ADCC targeting molecule species in a pharmaceutically acceptable medium. The invention further provides a method of inducing antibody-dependent cell cytotoxicity (ADCC) against a target cell. The method consists of contacting the target cell in the presence of effector cells with two or more ADCC targeting molecule species each selective for different antigens on the surface of the target cell. A method of treating a pathological condition characterized by aberrant cell growth is also provided. The method consists of administering an effective amount of two or more ADCC targeting molecules selective for different antigens expressed on the surface of cells mediating the pathological condition.

### DETAILED DESCRIPTION OF THE INVENTION

This invention is directed to methods of treating cancer and other aberrantly regulated cell proliferative conditions by utilizing the antibody-dependent cell cytotoxicity (ADCC) component of an individuals innate immune system against the pathological condition. The method targets two or more antibodies or other ADCC targeting molecules against different antigens expressed on the surface of the aberrantly proliferating cells as a means to destroy the pathological cells. The use of two or more ADCC targeting molecules toward different antigens increases the efficacy of ADCC therapy because it biases the system toward having at least one of the target antigens bound by an ADCC targeting molecule to effect cell destruction. An advantage of this method is that it alleviates problems associated with insufficient specificity or density of a target antigen for the pathological cell population and therefore allows ADCC killing to be effective against less abundant or less specific antigens. As the method is more efficacious, a formulation of two or more ADCC targeting molecules also can be less toxic by decreasing the total load of unbound molecules or of waste by-products in the liver. An additional advantage in targeting multiple different antigens is that it reduces or prevents the ability of a pathological cell population to escape destruction through down regulation of target antigen expression because such avoidance of ADCC effector mechanisms essentially would require concurrent down regulation of all targeted antigens.

In one embodiment, the invention is directed to the ADCC-mediated killing of prostate cancer cells using a pentameric antibody specific for two prostate cell antigens. The pentameric antibody is a hybrid molecule composed of chimeric IgG molecules each fused to IgM tails. The IgM tails allow assembly of the IgG fusions into an IgM-like pentameric structure upon addition of a J chain. The IgG portion of the fusion contains the F_{c} receptor binding domain which induces ADCC-mediated cell killing or destruction. Two species of antigen binding domains directed to different prostate cell markers are contained within the pentameric IgG molecule. This ADCC targeting molecule can be administered to individuals with prostate cancer to treat or reduce the severity of the disease.

As used herein, the term “antibody-dependent cell cytotoxicity” or “ADCC” is intended to mean the cell-mediated cytotoxicity component of the innate immune system. This cellular component of the innate defense mechanism results in destruction or engulfment of foreign particles, cells or host cells that aberrantly express inappropriate or foreign antigens. Cell or particle destruction includes, for example, killing by lysis, necrossis or toxicity. Engulfment includes, for example, phagocytosis. The term is intended to include innate defense mechanisms dependent on specific or selective opsonization of a particle or cell and induction of ADCC through F_{c} receptors. Therefore, induction of ADCC depends on the F_{c} receptor binding domain of an antibody constant region and molecules including the F_{c} receptor binding domain can be used to induced ADCC killing or engulfment. Specific examples of such molecules include antibodies and functional fragments which contain the F_{c} receptor binding domain as well as modified forms thereof and chimeric targeting molecules so long as such molecules contain an F_{c} receptor binding domain.

As used herein, the term “effector cell” when used in reference to ADCC, is intended to mean those cell types which contain F_{c} receptors. Such cell types can induce cell or particle destruction or engulfment through a F_{c} receptor-dependent manner and include, for example, natural killer (NK) cells and phagocytic cells. Specific examples of phagocytic cells include monocytes, macrophages, neutrophil, eosinophils and retinal epithelial cells. However, most cell types have phagocytotic activity as they express stimulatory F_{c} receptors and therefore are, or can be made to be ADCC effector cells of the invention. For example, NK cells contain stimulatory F_{c} receptors whereas macrophages contain both stimulatory and inhibitory F_{c} receptors. The term “effector cell” is similarly intended to include cells which have been modified so as to have altered ADCC cell destruction capability. Such modifications include, for example, increasing the expression of components of the ADCC defense system to enhance ADCC capability of effector cells increasing the binding activity or specificity of stimulatory F_{c} receptor binding domains and decreasing the binding activity or specificity of inhibitory F_{c} receptor binding domains. Components of the ADCC defense which can be altered include, for example, stimulatory F_{c} receptors, inhibitory F_{c} receptors, lytic enzymes, perforin and TNF. Modifications can be made by recombinant methods, natural mutation or other methods known to those skilled in the art.

As used herein, the term “ADCC targeting molecule” is intended to mean an antigen binding protein containing a F_{c} receptor binding domain capable of inducing ADCC. An ADCC targeting molecule is therefore at least bispecific, containing an antigen binding domain and a F_{c} receptor binding domain. The ADCC targeting molecule can include multiple valencies for either or both of the antigen binding domain or the F_{c} receptor binding domain. Additionally, an ADCC targeting molecule also can have multiple different antigen binding domains combined with a single or multiple copies of an F_{c} receptor binding domain or combined with different F_{c} receptor binding domains. The antigen binding domain or domains can be derived from essentially any molecule that has selective or specific binding activity to a target antigen so long as it can be fused or attached to one or more F_{c} receptor binding domains while still maintaining antigen binding activity. The F_{c} receptor binding domain can be derived from an antibody constant region of, for example, the IgG class, including subclasses IgG1, IgG3 and IgG4. Such F_{c} receptor binding domains can be used in their native form or the amino acid sequence can be modified so as to enhance or optimize the F_{c} receptor binding or ADCC activity. Moreover, the F_{c} receptor binding domains can be derived from constant regions which recognize either stimulatory or inhibitory F_{c} receptors. The F_{c} receptor binding domain is located within the hinge region of an antibody constant region where the cognate receptors bound by this domain include, for example, the FcγRI, FcγRIIA and FcγRIII. Therefore, ADCC targeting molecules include, for example, antibodies selective for a target antigen and functional variants thereof as well as fusion proteins and chemical conjugates containing both an antigen binding domain and a F_{c} receptor binding domain in functionally active forms.

As used herein, the term “species” when used in reference to an ADCC targeting molecule is intended to refer to the antigen reactivity of the targeting molecule or populations thereof. For example, antibody ADCC targeting molecules that have the same antigen binding activity are considered to be the within same species whereas antibody ADCC targeting molecules that have different binding activity are distinct species. ADCC targeting molecule species can be determined by, for example, antigen binding reactivity as well as by sequence identity since those which fall within the same species will have like binding activity and sequence identity or three-dimensional, tertiary structure.

As used herein, the term “functional variant” when used in reference to an antibody ADCC targeting molecule is intended to mean any modified form of an antibody, including a fragment thereof, which retains a functional variable region antigen binding domain and a functional F_{c} receptor binding domain capable of inducing ADCC. Therefore, an antibody functional variant can include chimeric and humanized antibodies as well as truncated and deleted forms of the antibody so long as the modification does not prevent the antigen binding or ADCC inducing activity of the variant targeting molecule. Similarly, antibody functional variants also include F_{d}, F_{c}, Fab and F(ab)₂ fragments fused to F_{c} receptor binding domains as well as single chain antibodies and heavy and light chain variable regions which alone exhibit antigen binding activity so long as the are fused or attached to a F_{c} receptor binding domain capable of inducing ADCC.

Functional variants of antibody ADCC targeting molecules also include numerous antibody-like molecules well known to those skilled in the art, including for example, bispecific antibodies, immunoadhesions and bispecific immunoadhesions. Other antibody-like molecules known inn the art are similarly included within the term so long as they contain or can be made to contain a F_{c} receptor binding domain capable of inducing ADCC. The term is similarly intended to include functional variants of ADCC targeting molecules other than antibodies so long as they retain antigen binding activity of the parent antigen binding domain and F_{c} receptor binding activity sufficient to induce ADCC.

As used herein, the term “selective” when used in reference to the antigen binding activity of an ADCC targeting molecule is intended to mean that the targeting molecule exhibits preferential binding affinity or avidity toward the target antigen. An ADCC targeting molecule that exhibits selective binding affinity also does not substantially cross-react with non-target antigens. Selective binding includes specific binding activity such as when an ADCC targeting molecule does not measurably cross- react with non-target antigens.

As used herein, the term “antigen” is intended to mean a molecule which can be bound by the antigen binding domain of an ADCC targeting molecule and form a covalent or non-covalent complex. In the specific case of antibodies, a noncovalent complex is formed in the antigen combining site of a variable region of the antibody. Therefore, the term “cellular antigen” is intended to mean an antigen that's endogenous to a cell of a mammalian organism which exhibits innate defense mechanisms. Cellular antigens originate or derive from the mammalian organism or are encoded by the genome of the mammalian organism. Cellular antigens consist of all types of macromolecules and include, for example, proteins, glycoproteins, carbohydrate and lipid. The term “heterologous antigen” is intended to mean those antigens which are originally foreign to a mammalian organism. Heterologous antigens include, for example, macromolecules originating, derived from or encoded by the genome of pathogens such as viruses, bacteria and parasites. The terms “cellular antigen” and “heterologous antigen” are similarly intended to included inappropriate or aberrantly expressed antigens derived from or encoded by each of their respective mammalian cell or pathogen origins.

As used herein, the term “pathological condition”when used in reference to aberrant cell growth, is intended to mean a disease or abnormal condition, including an injury, of a mammalian cell or tissue. Therefore, the term “aberrant cell growth” is intended to refer to those diseases or abnormal conditions that result in unwanted or abnormal cell growth, viability or proliferation. Pathological conditions characterized by unwanted or abnormal cell growth include, for example, cancer and other neoplastic conditions, infectious diseases and autoimmune diseases. For example, cancer cells proliferate in an unregulated manner and consequently result in tissue destruction. Similarly, the proliferation of cells mediating autoimmune diseases are aberrantly regulated which results in, for example, the continued, proliferation and activation of immune mechanisms with destruction of the host's cells and tissue. The growth of cells infected by a pathogen are unwanted and abnormal due to the intrusion of the foreign organism, for example. Specific examples of cancer include prostate, breast, lung, ovary, uterus, brain and skin cancer. Specific examples of infectious diseases include DNA or RNA viral diseases, bacterial diseases, paracytic diseases whereas autoimmune diseases include, for example, diabetes, rheumatoid arthritis and multiple sclerosis.

By specific mention of the above categories of pathological conditions, those skilled in the art will understand that such terms include all classes and types of these pathological conditions. For example, the term cancer is intended to include all known cancers, whether characterized as malignant, benign, soft tissue or sold tumor. By exemplification, a list of known cancers is provided below in Table 1. Similarly, and by analogy to the classes and types of cancers shown in Table 1, the terms infectious diseases and autoimmune diseases are intended to include all classes and types of these pathological conditions. Those skilled in the art will known the various classes and types of infectious and autoimmune diseases.

As used herein, the term “treating” is intended to mean reduction in severity or prevention of a pathological condition characterized by aberrant cell growth. Reduction in severity includes, for example, an arrest or a decrease in clinical symptoms, physiological indicators, biochemical markers or metabolic indicators. Prevention of the disease includes, for example, precluding the occurrence of the disease or restoring a diseased individual to their prediseased state of health.

As used herein, the term “effective amount” is intended to mean an amount of two or more ADCC targeting molecules required to effect a decrease in the extent, amount or rate of spread of a pathological condition when administered to an individual. The term includes the amount of all species of targeting molecules, whether combined into a single multimeric or multispecific targeting molecule or each formulated as distinct molecules, required to effect a decrease in progression or persistence of a pathological condition. Therefore, proportional or disproportional amounts of ADCC targeting molecule species are included in the formulations of the invention so long as of all ADCC targeting molecule species when combined are sufficient to effect a decrease in progression or persistence of a pathological condition. The dosage of an ADCC targeting molecule required to be therapeutically effective will depend, for example, on the pathological condition to be treated, the affinity and avidity of the targeting molecules, the level of abundance and density of the cognate antigens as well as the weight and condition of the individual, and previous or concurrent therapies. The appropriate amount considered to be an effective dose for a particular application of the method can be determined by those skilled in the art, using the guidance provided herein. For example, the amount can be extrapolated from in vitro or in vivo ADCC assays as described below. One skilled in the art will recognize that the condition of the patient needs to be monitored throughout the course of therapy and that the amount of the composition that is administered can be adjusted according to the response of the therapy.

The invention provides a method of inducing antibody-dependent cell cytotoxicity (ADCC) against a target cell. The method consists of contacting the target cell in the presence of effector cells with two or more ADCC targeting molecule species each selective for different antigens on the surface of said target cell.

The methods of the invention employ the use of two or more ADCC targeting molecules against a target cell. The ADCC targeting molecules bind the target cell and effect its destruction through an ADCC-mediated pathway. Binding of the ADCC targeting molecules to the target cell marks that cell for recognition and destruction by effector cells. F_{c} receptor-mediated binding of the effector cells to the ADCC targeting molecule signals destruction of the target cell within the cell-cell complex. Either or all of the two or more ADCC targeting molecules can induce ADCC killing of the target cell.

The methods of the invention are applicable against target cells of all types for which elimination is desired. The target cells can be isolated such as in a substantially pure form, or they can be components of a mixed or heterogeneous population. The methods of the invention are equally applicable to cells which grow in suspension as they are to cells which grow in monolayers and to target cells which are components of tissues and organs. Therefore, the methods of inducing ADCC for elimination of a target cell are applicable to cells, cell populations and target cells within a tissue for use both in culture and in individuals.

Particular types of target cells include, for example, those cells that are aberrantly regulated or abnormal because the ADCC targeting methods of the invention can selectively identify and eliminate such cells from surrounding, normal cells within the population or tissue. Aberrantly regulated cells include, for example, cells that exhibit uncontrolled cell proliferation as well as cells that exhibit dysfunction in specific phases of the cell cycle, leading to altered proliferative characteristics or morphological phenotypes. Specific examples of aberrantly regulated cell types include neoplastic cells such as cancer and hyperplastic cells characteristic of tissue hyperplasia. Another specific example includes immune cells that become aberrantly activated or fail to down regulate following stimulation. Autoimmune diseases are mediated by such aberrantly regulated immune cells. Aberrantly regulated cells also includes cells that are biochemically or physiologically dysfunctional. Other types of aberrant regulation of cell function or proliferation are known to those skilled in the art and are similarly target cells of the invention applicable for destruction using the ADCC-mediated methods of the invention.

Similarly, abnormal cells include aberrantly regulated cells such as those described above, and additionally include, for example, cells that are infected with a pathogen. Infectious agents that render a cell abnormal include, for example, those agents that require host cell machinery for survival or propagation. For example, viruses infect cells and cause cancer. Included within such infectious agents are DNA viruses, RNA viruses and parasites. Specific examples of DNA viruses include adenoviruses and paruoviruses. Specific examples of RNA viruses include reoviruses, poliomyelitis, influenza and retroviruses. Parasites that utilize eukaryotic host cell machinery for survival or propagation include, for example, trypanosomes. The cells infected by these and other agents known in the art which utilize host cell machinery are rendered abnormal because they are compromised in normal cellular function which can manifest in morphological and biochemical changes. As such, these cells can be targeted for elimination within a culture or within an individual using the ADCC-mediated killing methods of the invention.

Target cells applicable for destruction using the ADCC-mediated methods of the invention additionally include infectious agents and pathogens that are autonomous, and therefore propagate independently of host cell machinery. Such pathogens can live in the extracellular space of tissues and organs where they invade and cause destruction of surrounding tissue. Pathogen target cells of the invention include, for example, bacteria, amoeba, and fungi.

All of the above aberrant and abnormal cell classifications and cell types exhibit undesirable physiological characteristics and phenotypes which can lead to unwanted cell growth and complications. As such, these aberrantly regulated or abnormal cells can be targeted for destruction using the ADCC-mediated targeting methods of the invention.

Effector cell function in ADCC killing is to destroy target cells once they are marked or tagged by a bound ADCC targeting molecule. Cell destruction can occur, for example, by lysis or phagocytosis. Effector cells which are capable of cell destruction by lytic means include for, example, natural killer (NK) cells, eosinophils, macrophages and neutrophils. In these specific examples, F_{c} receptor- dependent killing is effected by the induction of a combination of perforin, lytic enzymes or tumor necrosis factor (TNF) once the effector cell recognizes and binds the target cell-ADCC targeting molecule complex via its F_{c} receptor.

Effector cells that are capable of cell destruction by phagocytosis include essentially all types of eukaryotic, and particularly mammalian, cells having cell surface F_{c} receptors. Such effector cells additionally include those cells modified to express F_{c} receptors. Effector cells that have efficient F_{c} receptor-dependent phagocytic capabilities include, for example, macrophages, monocytes and neutrophils. However, essentially all other cell types similarly can be effector cells of the invention as most cells express or can be modified to express F_{c} receptors on their cell surface. For example, thyroid and bladder epithelial cells phagocytose erythrocytes in vivo when modified to express stimulatory F_{c} receptors. Effector cells other than those described above and their level of F_{c} receptor expression and type are known in the art and can similarly be used in the methods of the invention.

For example, one difference with respect to ADCC-mediated killing activity capacity and efficiency between cells is the level of Fe receptor expressed on the cell surface. Those effector cells that express higher levels of stimulatory F_{c} receptor correspondingly exhibit greater capacity and efficiency of ADCC-mediated killing. Effector cells can be used unaltered so as to effect cell destruction at their inherent capacity and rate, or alternatively, such cells can be modified by recombinant methods to express higher levels of one or more stimulating F_{c} receptors on their cell surface. Modified effector cells will exhibit a corresponding increase in ADCC-mediated killing capacity and rate commensurate with the increase in F_{c} receptor expression. Moreover, the level of F_{c} receptor expression can be mediated by, for example, stimulation or inhibition with cytokines and other compounds. Such compounds include, for example, adjuvants and γ-interferon. Therefore, efficient effector cells can be further augmented to achieve increased efficacy of target cell destruction and, when desired, effector cells having moderate or low level ADCC-mediated killing capabilities can be made to mediate ADCC killing at levels comparable or greater to effector cells exhibiting efficient ADCC-mediated activity.

The ADCC-mediated methods of the invention can be used to induce target cell destruction in a variety of different settings known to those skilled in the art. Such settings include in vitro, in vivo and in situ methods for elimination of a target cell within, for example, a cell culture, tissue, organ or individual. For example, target cells can be contacted with two or more ADCC targeting molecule species in the presence of effector cells added to a culture. One or more of the ADCC targeting molecule species will bind the target cells and induce the co-cultured effector cells to destroy and eliminate the target cells. In vitro methods of eliminating specific target cells is an effective method for removing contaminating cells and infectious particles to obtain a pure homogeneous or mixed population of cells. For example, primary cell cultures are generally contaminated with numerous cell types. Applying the methods of the invention in such an in vitro culture setting, the unwanted cell types can be targeted for destruction to isolate a primary cell culture.

Similarly, unwanted cell types within tissues and organs can be eliminated in either an in vitro or in situ setting using the ADCC-mediated targeting methods of the invention. Tissues, organs or subcomponents thereof can be cultured in the presence of effector cells and two or more ADCC targeting molecules selective for the unwanted cell type to achieve target cell-effector cell complex formation and subsequent destruction of the target cell population. As with the in vitro applications described above, applying the methods of the invention to cultures of tissues, organs and subcomponents thereof similarly is an effective means to purge them of unwanted or contaminated cells and infectious agents.

For in vitro settings employing the methods of the invention, the specific type of effector cell is unimportant so long as it expresses functional stimulatory F_{c} receptor on its cell surface and is capable of mediating ADCC killing. Therefore, the particular type of effector cell will depend on the intended purpose. For example, NK cells or efficient phagocytic cells can be employed if rapid cell destruction is desired. Additionally, combinations of effector cell, including all effector cell types, can be added to the culture to invoke rapid and efficient ADCC-mediated target cell killing. The number of effector cells needed to achieve a desired rate of elimination is well known to those skilled in the art and is also described further below.

The methods of the invention also are applicable in situ and in vivo for the destruction of unwanted cells in tissues, organs and localized regions within an individual. It is not necessary however to add effector cells to the targeted tissues and regions or to the individual when practicing the methods in situ or in vivo as effector cells are already present in the individual. Effector cells can nevertheless be added simultaneously, consecutively or sequentially with the ADCC targeting molecules if an increased efficiency or efficacy is desired. Those skilled in the art will know, or can determine, when the exogenous addition of effector cells will augment the elimination of unwanted target cells within the treated region or the individual. As with the applications described above, the specific type of exogenous effector cell is unimportant so long as it expresses functional F_{c} receptor on its cell surface and is capable of mediating ADCC killing.

ADCC targeting molecules of the invention can consist of a variety of different types of binding proteins. Each targeting molecule will contain at least two binding characteristics. One binding characteristic is the ability to bind an antigen on the target cell. The second binding characteristic is the ability to bind an F_{c} receptor on an effector cell. Binding of a cell surface stimulatory F_{c} receptor initiates ADCC and subsequent cell death. Therefore, ADCC targeting molecules of the invention are at least bifunctional in that they contain a functional antigen binding domain and a functional F_{c} receptor binding domain.

ADCC targeting molecules can exhibit single or multiple valencies for each of the two functional binding domains as well as contain additional binding characteristics. For example, ADCC targeting molecules can be bi- or multivalent for a target antigen, for a particular F_{c} receptor or both. Additionally, multiple different binding specificties for either or both of the antigen binding domain and the F_{c} receptor binding domain can be included in an ADCC targeting molecule of the invention. The inclusion of multiple different antigen binding domains in a targeting molecule provides the advantage of being able to target multiple target antigens with a single molecule. Similarly, the inclusion of multiple different F_{c} receptor binding domains provides the advantage of increasing the range, and therefore efficiency, of effector cells that can recognize marked target cells and effect their destruction. The inclusion of multiple different F_{c} receptor binding domains additionally allows for the stimulation of stimulatory F_{c} receptors and the inhibition of inhibitory F_{c} receptors with, for example, variant forms of inhibitory F_{c} receptor binding domains.

ADCC targeting molecules also can incorporate functional characteristics other than those needed for target and effector cell binding and induction of ADCC-mediated cell destruction. Such other functional domains can include, for example, cytokine and growth factors that augment immune responses and modulate cell regulation. Therefore, ADCC targeting molecules can be multi functional, containing multiple binding specificties for either or both of the antigen binding domain and the F_{c} receptor binding domains. ADCC targeting molecules can additionally contain binding specificties and functional characteristics that are independent of target cell binding and ADCC induction.

A specific example of an ADCC targeting molecule is an antibody, including polyclonal antibodies and monoclonal antibodies. Antibody variable region domains bind antigen while the F_{c} receptor binding domain is located within the constant region. Human antibody subclasses capable of binding an F_{c} receptor and inducing ADCC are within the IgG class. Without modifications, antibodies generally are bivalent for a particular antigen. However, and as described below, numerous alterations and variants of antibodies are well known to those skilled in the art that an antibody ADCC targeting molecule can be take on many different forms and functional equivalents. Therefore, antibody ADCC targeting molecules of the invention include, for example, polyclonal antibodies, monoclonal antibodies as well as recombinant versions and functional fragments thereof so long as they retain both an antigen binding domain and a F_{c} receptor binding domain.

Recombinant versions of antibody targeting molecules include a wide variety of constructions ranging from simple expression and co-assembly of encoding heavy and light chain cDNAs to speciality constructs termed designer antibodies. Recombinant methologies, combined with the extensive characterization of polypeptides within the immunoglobulin superfamily, and particularly antibodies, provides the ability to design and construct a vast number of different types, styles and specificties of binding molecule derived from immunoglobulin variable and constant region binding domains. Specific examples include chimeric antibodies, where the constant region of one antibody is substituted with that from another antibody, and humanized antibodies, where the complementarity determining regions (CDR) from one antibody are substituted with those from another antibody. Chimeric antibodies are useful as ADCC targeting molecules because F_{c} receptor binding can be conferred onto a non-F_{c} receptor binding antibody subclass which has a desired binding specificity. For example, an IgM variable region or F(ab)₂ fragment can be fused to an IgG subclass to confer ADCC induction onto the IgM binding specificity. Similarly, humanized antibodies are useful as ADCC targeting molecules because essentially any antigen binding specificity can be conferred onto a human antibody framework so as to avoid host immune responses against the antibody ADCC targeting molecule when used therapeutically.

Other recombinant versions of antibody ADCC targeting molecules include, for example, functional antibody variants where the variable region binding domain or functional fragments which maintain antigen binding is fused with an F_{c} receptor binding domain from the antibody constant region. Such variants are essentially truncated forms of antibodies which remove regions non-essential for antigen and F_{c} receptor binding. Truncated variants can be have single valency, for example, or alternatively be constructed with multiple valencies depending on the application and need of the user. Additionally, linkers or spacers can be inserted between the antigen and F_{c} receptor binding domains to optimize binding activity as well as contain additional functional domains fused or attached to them to effect biological functions other than ADCC-mediated killing. Other variants can include, for example, modifying the F_{c} receptor binding domain to increase binding activity for stimulatory F_{c} receptors or to decrease binding activity to inhibitory F_{c} receptors. Those skilled in the art will know how to construct variant antibody ADCC targeting molecules in light of that known in the art for antibody engineering and given the guidance and teachings herein. A description of recombinant antibodies, functional fragments and variants and antibody-like molecules can be found, for example, in “Antibody Engineering,” 2nd Edition, (Carl A. K. Borrebaeck, Ed.) Oxford University Press, New York,(1995).

Additional functional variants of antibodies that can be used as ADCC targeting molecules of the invention include antibody-like molecules other than antigen binding-F_{c} receptor binding domain fusions. For example, antibodies, functional fragments and fusions thereof containing a F_{c} receptor binding domain can be produced to be bispecific in that one variable region binding domain exhibits binding activity for one antigen and the other variable region binding domain exhibits binding activity for a second antigen. Such bispecific antibody ADCC targeting molecules can be advantages in the methods of the invention because a single bispecific antibody will contain two different target antigen binding species. Therefore, a single molecular entity can be administered to achieve ADCC targeting to two or more different antigens on the target cell surface.

Immunoadhesions and bispecific immunoadhesions are further examples of antibody functional variants that can be used as ADCC targeting molecules of the invention. Immunoadhesions are antibody-like molecules that combine the binding domain of a non-antibody polypeptide with the effector functions of an antibody of an antibody constant domain. The binding domain of the non-antibody polypeptide can be, for example, a ligand or a cell surface receptor having ligand binding activity. Immunoadhesions for use as ADCC targeting molecules should contain at least the F_{c} receptor binding effector functions of the antibody constant domain. Specific examples of ligands and cell surface receptors that can be used for the antigen binding domain of an immunoadhesion ADCC target molecule include, for example, a T cell receptor, selecting, homing receptors, NP receptor, TNF receptor, interferon γ receptor, CD4, CD44, CD28, CD22, CTLA-4, TNF, and interferons. Other ligands and ligand receptors known in the art can similarly be used for the antigen binding domain of an immunoadhesion ADCC targeting molecule of the invention.

Multivalent and multispecific immunoadhesions can -be constructed for use as ADCC targeting molecules of the invention. Specific examples include bivalent and bispecific immunoadhesions where the two variable region arms of an antibody are replaced with binding protein ligands or with ligand binding domains of receptors. Combinations of antibody variable region binding domains, binding protein ligands, and ligand binding domains of receptors can similarly be constructed to form hybrid antibody-like ADCC targeting molecules. Those skilled in the art will know how to construct these and other multivalent and multispecific antibody-like molecules for use as ADCC targeting molecules of the invention given the teachings and guidance provided herein. The construction of bispecific antibodies, immunoadhesions, bispecific immunoadhesions and other heteromultimeric polypeptides which can be used as ADCC targeting molecules of the invention is the subject matter of, for example, U.S. Pat. Nos. 5,807,706 and 5,428,130.

Multivalent and multispecific antibodies other than the bispecific variants described above can similarly be used as ADCC targeting molecules of the invention. For example, a pentameric antibody can be produced which contains binding specificties for between one and ten target antigens within the same molecular entity. Pentameric antibodies can be assembled from IgM classes of antibodies. In the presence of a J chain, IgM antibodies co-assemble into a pentameric structure. If the antibodies are bivalent and unispecific, the assembled structure will contain ten binding domains all of the same specificity. However, by assembling a bispecific antibody species, five binding domains for each of the two binding specificties can be produced in a single pentameric structure. Similarly, by co-assembly of single binding species, bispecific species and combinations thereof, pentameric antibody variants can be produced that contain all combinations between one and ten different binding specificties within a total of ten binding domains.

For use as an ADCC targeting molecule, the pentameric antibody variants described above will have to contain at least one F_{c} receptor binding domain within the constant region of one of the antibody species. Inclusion of an F_{c} receptor binding domain can be accomplished by producing a fusion between an IgM J chain binding domain, which is needed for pentameric assembly, and a F_{c} receptor binding domain of an IgG subclass. The J chain binding domain resides at the C-terminal end of the constant region and the F_{c} receptor domain resides N-terminal to the analogous region in the corresponding IgG molecule. Fusion of these two domains will impart IgM pentameric assembly function onto F_{c} receptor binding domain yielding a hybrid constant exhibiting both functions. Such pentameric antibodies also can be modified by, for example, inclusion of cystine residues to further stabilize the structure through interchain disulfide bonds. Although at least one F_{c} receptor binding domain is needed within the pentameric assembly, the greater number of F_{c} binding domains contained within the pentameric structure, the more efficient a particular variant will be at inducing ADCC-mediated killing. Therefore, formation of a pentameric ADCC targeting molecule where all subunit ADCC targeting molecules contain F_{c} receptor binding domains will be the most efficient at inducing ADCC cell destruction.

ADCC targeting molecules other than those exemplified above are known and also can be used in the methods of the invention. For example, a diabody is a bivalent or bispecific recombinant antibody-like fragment which is a non-covalent dimer of two single-chain Fv molecules having relatively short linkers such that the V_{L} and V_{H} domains of one chain pair with the complementary V_{H} and V_{L} domain of the second chain to form two antigen binding sites. Diabodies can be ADCC targeting molecules when one of the variable region binding domains is specific for a F_{c} receptor and can induce ADCC by binding an effector cell. Diabodies are well known in the art and can be found described in, for example, Holliger et al., Proc. Natl. Acad. Sci. USA 90:6444-6448 (1993)and Carter and Merchant, Curr. Olin. Biotech. 8:449-454 (1997). Anti-F_{c} receptor antibodies, functional fragments and variants thereof are similarly well known in the art and are the subject matter of U.S. Pat. No. 5,837,243.

Other antibody-like variants known in the art which can be used, or modified for use as ADCC targeting molecules of the invention include minibodies and miniantibodies. A minibody is a bivalent or bispecific recombinant antibody-like fragment produced as a covalent or non-covalent dimer of two polypeptides which each contain a single chain Fv molecule fused to an antibody domain corresponding to the third constant region of an antibody heavy chain. Minibodies can be found described in, for example, Holliger and Winter, Cur. Op. in Biotechnology 4:446-449 (1993) and Carter and Merchant, supra. A miniantibody is a bivalent or bispecific antibody fragment that is also a dimer of two single chain Fv fragments. However, each of the single chain Fv fragments are linked via a hinge region to a dimerization domain such as a leucine zipper or helix-turn-helix motif. Miniantibodies can be found described in, for example, Pack et al., Biotechnol. 11:1271 (1993) and Holliger and Winter, supra. These molecules and others can be modified to contain one or more F_{c} receptor binding domains and as such, applicable for use as an ADCC targeting molecule of the invention.

Given the guidance and teachings herein, ADCC targeting molecules other than those described above, are known or can be constructed by those skilled in the art. Such molecules should have an antigen binding domain and an F_{c} receptor binding domain capable of inducing ADCC target cell killing when bound by an effector cell. Binding molecules containing these two functions are applicable for use in the methods of the invention. Methods for producing ADCC targeting molecules are well known in the art. Such methods can be found described in the references cited above as well as in Harlow and Lane, Using Antibodies: A Laboratory Manual, Cold Spring Harbor Laboratory Press, New York, 1999.; Sambrook et al., Molecular Cloning: A Laboratory Manual, Cold Spring Harbor Laboratory, New York (1992) and in Ansubel et al., Current Protocols in Molecular Biology, John Wiley and Sons, Baltimore, Md. (1989).

Therefore, the invention provides ADCC targeting molecules that are multivalent, multispecific or combinations thereof. Pentameric and antibody functional variant ADCC targeting molecules are also provided. The ADCC targeting molecules can include a single antigen binding domain species, two different antigen binding domain species, three or more and up to ten different binding domain species.

Antigens selected for targeting using the ADCC-mediated killing methods of the invention are cell surface polypeptides in order to be recognized and bound by an ADCC targeting molecule. The cell surface polypeptides selected for targeting are characterized by selective expression on the target cell type or on the tissue desired to be eliminated by the methods of the invention. Cell surface polypeptides can be, for example, membrane proteins, transmembrane proteins as well as intercellular vesicle membrane proteins that cycle to the cell surface and are therefore capable of recognition by an ADCC targeting molecule of the invention. For example, it has become recognized that in aberrantly regulated cells such as cancer, membrane trafficking is disrupted such that lysosomal and other vesicular membrane proteins become more abundant on the cell surface (Harter and Mellman, J. Cell. Biol. 117:311-325 (1992)).

Cell surface polypeptides that are antigens applicable for targeting using the methods of the invention can additionally originate from cellular sources, heterologous sources or both. For example, aberrant cell growth such as cancer is characterized by phenotypic changes that includes altered cell surface expression of endogenous genes. One or more of such endogenous genes can be targeted for elimination of these cells. Additionally, cancer and other aberrant cell types can include altered cell surface expression of a heterologous gene originating from, for example, a virus or other infectious agent. Similarly, such heterologous antigens that are characteristic of the aberrant target cell type can be used as antigens in the methods of the invention. As described further below, cellular and heterologous cell surface antigens specific or selective for a particular target cell type and are characteristic of such cell types in the absence of a correlation with a particular aberrant phenotype can additionally be used as target cell antigens. Therefore, depending on the application, antigens useful in the methods of the invention can be specific or selective for the target cell type or additionally with the aberrant cell phenotype.

Specific examples of cell surface cellular antigens that can be used as target antigens in the methods of the invention include, for example, Lewis-X, CEA, Her-2/neu receptor, CD-40, GD-3 and TAG-72. Specific examples of cellular antigens for pathological conditions other than cancer include, for example, T cell receptors (TCR) for autoimmune diseases. Specific examples of cell surface heterologous antigens that can be used as target antigens in the methods of the invention include, for example, DNA and RNA virus antigens and bacterial antigens. Other cell surface antigens that exhibit selective expression on the target cell or tissue are similarly applicable for use in the methods of the invention. Such other cell surface antigens include a wide range of target antigens and levels of expression. Those skilled in the art will know, or can determine, which of such antigen can be targeted using the methods of the invention.

In addition to known target cell antigens, those skilled in the art additionally can identify new antigens on a target cell or tissue to be eliminated. Methods well known in the art can be rapidly applied for identification of target antigens selective for the target cell or tissue to be eliminated. Such methods generally involve probing the target cell for differences in expression of cell surface markers compared to non-target cells. Differences in expression can be probed either at the nucleic acid level or, alternatively, at the polypeptide level. Methods for identifying new target antigens can be found described in, for example, Harlow and Lane, supra, and in Ansubel et al., supra. These methods can additionally be used for further characterizing known target antigens. Additionally, proteomics also can be used for identifying or characterizing new antigens on a target cell or tissue to be eliminated.

Moreover, selection of appropriate cell surface target antigens can be made in the absence of knowledge of the physical characteristic of the target antigen. Instead, it is sufficient to have an ADCC targeting molecule that is selective for the target cell or tissue. For example, there are numerous ADCC targeting molecules, such as antibodies, that have been characterized as selective for a particular target cell type or tissue without isolation or characterization of the cognate antigen. Knowledge of the binding selectivity of the ADCC targeting molecule toward the target cell is sufficient to identify that ADCC targeting molecule as one that can be used in the methods of the invention. Therefore, it is sufficient that the ADCC targeting molecule have selective binding activity toward the target cell or tissue in order to select an appropriate cell surface target antigen applicable in the methods of the invention.

One advantage of the methods of the invention employing two or more ADCC targeting molecule species each selective for different antigens on the target cell surface is that stringent criteria previously required for high target antigen density or unique cell type specificity are unnecessary. These criteria are circumvented because the addition of two or more different ADCC targeting molecules preferentially biases the system toward formation of a target cell-effector cell complex with at least one of the ADCC targeting molecule species. Increasing the number of different ADCC targeting molecule binding species against different antigens on a particular target cell results in a corresponding increase in the efficiency and efficacy of ADCC-mediated target cell death. Therefore, the methods of the invention can be effectively used against target antigens ranging from highly abundant cell surface levels to moderately levels to low levels, including rare polypeptide species. Those skilled in the art will know, or can determine, the number of ADCC targeting molecules to use, what level of expression and what cell type specificity of target antigen is sufficient to effectively destroy the target cell using the methods of the invention.

The selection of target cell antigens to direct ADCC-mediated killing can depend, for example, on the number and type of ADCC targeting molecules, the selectivity of the ADCC targeting molecules for each of the selected target antigens and the abundance of the targeted antigens. Those skilled in the art will appreciate given the teachings and guidance provided herein that it is not necessary to optimize each of the above parameters in order to achieve effective ADCC-mediated cell destruction. Instead, the above parameters can fluctuate, in a compensatory manner, so as to achieve effective ADCC induction. For example, two or more ADCC targeting molecules selective for two different moderately abundant target antigens will achieve selective ADCC destruction of the target cell within a heterogenous population. However, if it is desired to select a lower abundant target antigen, compensation to maintain the efficiency of ADCC killing can be achieved, for example, by selecting a higher abundant target antigen as the second target or by including a third ADCC targeting molecule selective to yet a different target antigen. ADCC targeting molecules greater than three also can be employed. Similarly, compensation also can be achieved by, for example, increasing the valency of the ADCC targeting molecule, such as using a pentameric antibody variant or by increasing the affinity of one or more of the ADCC targeting molecules.

In like manner, if higher abundant antigens are selected, then the number, selectivity and valency of the ADCC targeting molecules can be correspondingly lower compared to moderate or lower abundant antigens. Various combinations and permutations other than those exemplified above also can be employed for modulating the number and type of ADCC targeting molecule, the selectivity of the ADCC targeting molecule and the cell specificity of the antigen to achieve effective and selective destruction of target cells in light of the selection of particular target cell antigens. Therefore, the methods of the invention are applicable to the elimination of target cells even in the absence of the availability of densely arranged, abundant cell surface markers because the number of antigen targets and the above describe modulations of ADCC targeting molecule component can be adjusted to compensate for undesired characteristics of the target antigens so as to achieve a desired ADCC-mediated target cell killing result.

Additionally, the number and type of ADCC targeting molecule or the binding selectivity can be modulated or the target antigen abundance can be selected so as to increase or optimize ADCC induction. As with the modulation of these components allowing for compensatory adjustments with respect to each other described above, the overall effectiveness of the ADCC killing methods of the invention can be further altered by their subsequent modulation of any or all of these components to achieve a desired result. Alteration of the overall effectiveness can be either to achieve increased or decreased ADCC killing efficiency or efficacy and will depend on the need and application of the user. For example, increased effectiveness can be achieved by including three or more ADCC targeting molecules, selecting higher abundant target antigens, increasing the valency of the ADCC targeting molecules, increasing the affinity or specificity of the ADCC targeting molecules or any combination thereof. Similarly, to decrease the overall effectiveness, one or more, including combinations thereof, the above components can be selected for the opposite activity. Using such an approach, combined with the teachings and guidance herein, the ADCC-mediated methods of the invention can be improved, optimized and tailored for a particular application or different applications.

Methods for assessing the ability of a particular combination of ADCC targeting molecule species or type, binding selectivity and target antigen which can effectively mediate ADCC killing are well known in the art and are described further below. Such methods are similarly applicable for determining the effect on ADCC-mediated killing of various combinations and permutations selected to increase or optimize ADCC induction. For example, given a particular target cell, population or tissue that is to be eliminated, the user can place a sample in one of the assays described below and measure the ability of ADCC-mediated destruction of the target cells in the presence of two or more ADCC targeting molecule species selective for different antigens on the target cell surface. The ability of ADCC-mediated killing can be further measured with the substitution of, for example, a higher affinity or greater number or valency of ADCC targeting molecules until a desired level of target cell destruction is achieved. Such substitutions can be made stepwise, simultaneous or in parallel to obtain the desired level. Given the teachings and guidance provided herein, the selection of two or more different target antigens that induce ADCC-mediated destruction of target cells will be achieved.

In a similar manner, to increase or optimize the efficiency or efficacy of ADCC-mediated killing methods of the invention, the user can follow these same procedures with essentially any combination of target antigens, number of ADCC targeting molecules species, type of ADCC targeting molecule, binding selectivity and cell specificity. Therefore, to obtain a formulation of two or more ADCC targeting molecules that is effective to selectively destroy a target cell as well as to modulate the killing effectiveness by, for example, increasing ADCC efficiency or optimizing efficacy, all that is necessary is to make and test such formulations or modifications thereof in methods well known in the art for determining ADCC cell destruction.

The advantage of being able to optimize ADCC induction efficiency and efficacy is appreciated not only in application of the methods to a particular pathological condition, but also in context of the application of the methods of the invention to a wide range of different target cells and pathological conditions. The ADCC-mediated methods of the invention are applicable to essentially any target cell or population thereof which contains two or more different cell surface antigens. Therefore, depending on the type of target cell, population or tissue that is being eliminated, optimization can be different than increasing the efficiency of ADCC killing. Specifically, optimization refers to adjusting the level of ADCC-mediated killing to achieve a predetermined result and can include, for example, increasing the efficiency of ADCC killing, increasing the efficacy of target cell elimination or modulating the rate of target cell elimination so as to accomplish that result.

For example, high affinity ADCC targeting molecules can achieve both efficient ADCC killing and efficacious elimination of the target cell when used against target cells in suspension, such as cultured cells or cells within the hematopoietic system, as well as against cells in soft tissue and organs. However, lower affinity ADCC targeting molecules, although less efficient at mediating ADCC killing, can nevertheless achieve efficacious elimination of, for example, solid tumors as target cells because they can more effectively penetrate the tumor. Other applications amenable to modulating the efficiency, efficacy or rate of ADCC-mediated killing to achieve a desired result are known to those skilled in the art and can be accomplished using the method of the invention.

For target cell destruction, the target cells are contacted with an amount of ADCC targeting molecule species effective to induce ADCC-mediated killing by the effector cells. An effective amount is an amount of all ADCC targeting molecule species contacted with the target cell which is sufficient to effect a measurable decline in target cell number or rate of proliferation. As described further below, measurement of target cell decline or rate of proliferation can be by direct or indirect means, including for example, the measurement of physiological characteristics or clinical symptoms. Therefore, an effective amount of ADCC targeting molecule species is an amount that can range from a level necessary to either reduce the number or rate of proliferation of target cells to a level that can eliminate the target cells within a cell population, tissue, organ or individual. The effect of any of these ranges will be a reduction in the severity or prevention of unwanted characteristics or of pathological conditions due to the target cells within the treated cell population, tissue, organ or individual.

The methods of the invention employ the use of two or more ADCC targeting molecule species. An amount effective to induce ADCC-mediated killing by effector cells can be any ratio of the two or more ADCC targeting molecule species so long as the total amount of species is sufficient to induce ADCC-mediated killing in the presence of effector cells. Therefore, the molar amount of each species within an ADCC targeting molecule formulation can be different so long as the combined molar amounts for all ADCC targeting molecule species is sufficient to induce ADCC-mediated killing. For the specific example of two ADCC targeting molecule species, the formulation can have equal ratios of each species or one species can represent as small as one molar percent or less and one species can represent as large as ninety-nine molar percent or greater within the formulation. All ratios in between these percentages can similarly be used in a formulation of an effective amount of an ADCC targeting molecule species so long as the combined ADCC targeting molecule species is effective at inducing ADCC killing of target cells. In like manner, formulations can be composed of essentially all combinations of molar ratios where there are greater than two ADCC targeting molecule species. Therefore, formulations having different ratios of three, four or five or more ADCC targeting molecule species are similarly effective at inducing ADCC- mediated killing of target cells.

An advantage of being able to modulate the ratios of ADCC targeting molecule species within a formulation is that it allows for greater flexibility and a broader range of selection for target antigens and ADCC targeting molecules. For example, having the capability of altering one or more components allows for compensation of ADCC targeting molecule amounts due to, for example, difference in binding affinity toward target antigens. Therefore, ADCC targeting molecule species can be formulated which are directed to various combinations of target antigens without any, or only minimal increases in total amount of ADCC targeting molecule protein within different formulations.

An amount effective to induce ADCC-mediated killing also can be modulated by, for example, altering or modifying the type of ADCC targeting molecule, its configuration, valency or affinity toward one or more target antigens or toward the F_{c} receptor. Such modifications allow for modulation of the required amount of ADCC targeting molecule species which is sufficient in the methods of the invention because they can affect the ADCC-mediated killing activity induced by the targeting molecule. Modifications which increase ADCC-mediated killing activity allow for a corresponding decrease in the relative amount of one or more ADCC targeting molecule species within a formulation while still maintaining a similar level of ADCC-mediated killing activity as the unmodified formulation. Conversely, modifications that decrease ADCC-mediated killing activity require a corresponding increase in the relative amount of one or more ADCC targeting molecule species within the formulation to maintain activity. Alternatively, a compensatory modification in a second ADCC targeting molecule species that increases its activity can achieve the same effect.

For example, where the ADCC targeting molecules species are different ADCC targeting molecules, the relative amount of one or more species within the formulation can be modulated by increasing the valency or affinity of the ADCC targeting molecule species toward its antigen. Increasing valency or affinity of the ADCC targeting molecule species enables the use of lower concentrations of that species within the formulation while still maintaining the same level of ADCC-mediated killing. Reducing the relative amount of one or more species within a particular formulation having sufficient ADCC-mediated killing activity has the advantage of being able to reduce the total amount of ADCC targeting molecule polypeptide while maintaining a comparable level of killing activity.

Similarly, the relative amount of one or more species within the formulation required to achieve effective ADCC-mediated killing activity can be modulated by altering the configuration of the ADCC targeting molecule from, for example, single binding species to molecules that exhibit binding activity for more than one of the target antigens. For example, both single and multivalent ADCC targeting molecules can be linked chemically or recombinantly to produce heteromeric and other multimeric forms of two or more binding species of ADCC targeting molecules. When such mulimeric ADCC targeting molecules are contacted with target cells, the linkage of multiple antigen binding species in a single complex ensures that when one ADCC targeting molecule species is bound to a target antigen, the other linked species are similarly in close proximity and also capable of binging the target cell. Incorporation of all ADCC binding molecule species into a single multispecies molecule essentially reduces the association kinetics for all ADCC targeting molecule species to a single bimolecular reaction, having the effect of increasing the apparent effective concentration at the target cell surface of the linked ADCC targeting molecule species. Therefore, incorporating multiple ADCC targeting molecule species into a single molecular entity allows for a corresponding reduction in the relative amount of one or more ADCC targeting molecule species within a formulation without substantially affecting the ADCC-mediated killing activity of the original formulation. Specific examples, of multimeric ADCC targeting molecules include bispecific antibodies, bispecific immunoadhesions, and pentameric antibodies having between two and ten different binding species.

In addition, multimeric ADCC targeting molecules can be produced to have flexible or constrained configurations to further modulate the effectiveness of ADCC targeting molecule species relative to each other and therefore, to modulate the relative amount of one or more species within a formulation sufficient to induce ADCC-mediated killing. For example, where the abundance or density of the different target antigens are sufficiently distinct, multimeric ADCC targeting molecules having flexible conformations can be more effective to allow freedom of movement between each of the different ADCC targeting molecule species, enabling access of the untethered species to a larger cell surface region following binding of one species within the multimeric complex. Conversely, where the abundance or density of the different target antigens are similar, constrained conformations can be more effective to increase the availability of all binding species within a localized area of the target cell following binding of one of one species within the multimeric complex to its antigen.

The relative amount of one or more species within a formulation effective to induce ADCC-mediated killing can additionally be modulated by substituting different types of ADCC targeting molecules within the formulation. In therapeutic settings for example, humanized forms of antibodies and other types of ADCC targeting molecules are longer lived compared to their non-human or chimeric counterparts and therefore will persist longer in the circulatory system and in tissues of the individual longer than non-human forms. Humanized forms of ADCC targeting molecules will therefore enable the use of lower amounts of one or more the ADCC targeting molecule species in the formulation.

ADCC targeting molecules having different types of F_{c} receptor binding domains also can be used to modulate the relative amount of one or more species within a formulation. For example, F_{c} receptor binding domains exhibiting increased stimulatory activity or F_{c} receptor binding domains exhibiting inhibitory activity can be used in various combinations to enhance the capacity and efficiency of ADCC-mediated killing of effector cells. Stimulatory F_{c} receptor binding domains can be added, for example, and inhibitory F_{c} receptor binding domains can be deleted or substituted with stimulatory domains from one or more ADCC targeting molecule species to enhance stimulation or decrease inhibitory regulation of ADCC-mediated killing. Similarly, combinations of stimulatory and inhibitory F_{c} receptor binding domain can be used together with different ADCC targeting molecule species within a formulation to further augment the effective ADCC activity of effector cells. As with the previous modulations of ADCC targeting molecules, the inclusion of stimulatory or removal of inhibitory F_{c} receptor binding domains will corresponding allow for a reduction in the amount of one or more ADCC targeting molecule species within a formulation to effect a comparable level of ADCC-mediated killing as the original formulation.

With any of the above modifications, the amount which is effective to induce ADCC-mediated killing can be further modulated by, for example, altering either or both of the F_{c} receptor binding domain or the target antigen binding domain of the ADCC targeting molecule. As described above, different functional types of F_{c} receptor binding domains can be used in various ways to augment ADCC-mediated killing. The affinity or specificity of these functionally different F_{c} receptor binding domains can be additionally modified to further enhance the ADCC stimulatory effects or suppress the ADCC inhibitory effects mediated by these domains and their cognate F_{c} receptors. For example, ADCC-mediated killing can be increased through modification in stimulatory F_{c} receptor binding domains of ADCC targeting molecule species which increase the F_{c} receptor binding affinity or specificity. Conversely, ADCC-mediated killing also can be increased through modification in inhibitory F_{c} receptor binding domains which decrease or prevent the F_{c} receptor binding of the ADCC targeting molecule.

Combinations of increasing the activity of stimulatory F_{c} receptor binding domains and decreasing or preventing the activity of inhibitory F_{c} receptor binding domains also can be produced to increase ADCC-mediated activity. Such combinations can be produced within the same or different ADCC targeting molecules within a formulation and include, for example, the incorporation of both modified stimulatory and inhibitory F_{c} receptor binding domains within a multimeric ADCC targeting molecule as well as the inclusion of distinct F_{c} receptor stimulatory and F_{c} receptor inhibitory ADCC targeting molecules or species within a formulation. Given the teachings and guidance described herein, other combinations also exist which can be produced by those skilled in the art for use in the methods of the invention. Therefore, modification F_{c} receptor binding domains allows for modulation of ADCC-mediated killing activity, and consequently, for modulation of the relative amount of one or more ADCC targeting molecule species in a formulation which is sufficient to induce cell destruction using the methods of the invention.

Similarly, the affinity or specificity of the target antigen binding domain of an ADCC targeting molecule also can be modified to further augment its binding activity and therefore its ability to induce ADCC-mediated killing. Modifications can be made to alter the binding affinity, or alternatively, to alter the rate of association or the rate of disassociation without substantial change in the overall binding affinity. Increases in the binding affinity or rate of association enhance the binding strength or specificity of the target antigen and therefore increase the ADCC-mediated killing activity of the ADCC target molecule species. Similarly, decreases in the rate of disassociation increase the life of the antigen-target molecule complex with a corresponding increase in the ADCC-mediated killing activity of the ADCC target molecule species. Such modifications allow for modulation of the relative amount of one or more ADCC targeting molecule species in a formulation which is sufficient to induce ADCC-mediated killing using the methods of the invention.

The teachings described above have been directed to various alterations and modifications of ADCC targeting molecule type, configuration, valency and affinity toward target antigens or F_{c} receptors which can be employed to modulate the ADCC-mediated killing activity of ADCC targeting molecules and targeting molecule species of the invention. Such modulations of activity allow for a wide range of combinations and permutations of ADCC targeting molecule formulations sufficient to induce ADCC-mediated killing in the methods of the invention. For example, ADCC targeting molecules can be produced that combine two or more of the previously described modifications to achieve even additional increases in ADCC-mediated killing activity when compared to a single modification alone. Similarly, ADCC targeting molecules can be produced that incorporate most or all of the previously described modifications to yield an even greater corresponding increase in ADCC-mediated killing activity of the ADCC targeting molecule. Such multiple combinations of ADCC targeting molecule modifications can be incorporated in, for example, single ADCC targeting molecule species, single ADCC targeting molecules, including multimeric and multispecies targeting molecules, and in ADCC targeting molecule formulations containing different combinations of modified ADCC targeting molecules and species thereof. Given the teachings and guidance provided herein, those skilled in the art will be able to select the appropriate modifications beneficial for a particular application of the methods of the invention.

Moreover, the various alterations and modifications of ADCC targeting molecules describe above also can be made to some or all of the ADCC targeting molecules and species employed in the methods of the invention. Incorporation of such modifications into multiple, including all, ADCC targeting molecules or species enables the modulation of the total ADCC-mediated killing activity of a particular formulation. Therefore, in addition to modulating amount of one or more ADCC targeting molecule species within a formulation, one or more modifications of multiple ADCC targeting molecules or species are additionally beneficial to enhance or optimize the efficiency or efficacy of the formulation's killing activity. An increase in the efficiency or efficacy will result in a corresponding decrease in the the total amount of ADCC targeting molecule polypeptide within a formulation which is necessary to mediate ADCC killing of target cells.

As described previously, in some applications the ADCC-mediated killing activity of two or more ADCC targeting molecules can be less than the maximal obtainable to achieve optimal efficacy in the methods of the invention. Therefore, the modifications of ADCC targeting molecules enabling modulation of relative and total amounts of ADCC targeting molecules effective to induce ADCC-mediated killing described previously in reference to increasing the ADCC-mediated killing activity of an ADCC targeting molecule species or formulation, can be applied analogously to achieve the opposite results. Specifically, the ADCC targeting molecule type, configuration, valency or affinity toward one or more target antigens or toward the F_{c} receptor can be inversely modified compared to that described above to decrease the ADCC-mediated killing activity of one or more ADCC targeting molecule species or total activity of a formulation. In such situations, the decreased ADCC-mediated killing activity will result in less efficient, but more efficacious elimination of target cells.

Numerous variations other than those described above exist for modulating the ADCC-mediated killing activity and therefore the amount of ADCC targeting molecule species effective to induce ADCC-mediated killing. Such variations include additional modifications other than those described above to the ADCC targeting molecule type, configuration, valency or affinity toward one or more target antigens or toward the F_{c} receptor as well as other modifications to ADCC targeting molecules known to those skilled in the art. Those skilled in the art will known what modification, or combination of modifications, will be desirable depending on a particular application. Therefore, the methods of the invention provide a wide range of choices in ADCC targeting molecules and formulations sufficient to induce ADCC-mediated killing of target cells.

Methods for making and modifying ADCC targeting molecules are well known in the art and have been described previously. Methods for making variants of ADCC targeting molecule species that exhibit altered binding activity or specificity for target antigen or for F_{c} receptors are similarly well known to those skilled in the art. For example, the binding affinity of either or both of the antigen binding domain or the F_{c} receptor binding domain can be modified by site directed mutagenesis of preselected amino acid residues. Amino acid changes can be made at single or multiple locations to alter the binding affinity of the modified domain. Such methods of mutagenesis are well known in the art.

Preselected changes can be made in the active binding region to directly affect binding affinity of the domain, or, such changes can be made in non-binding domain locations to indirectly alter the binding affinity of the domain, such as by, for example, a change in conformation of the binding domain. Additionally, one or more amino acid substitutions can be made at a preselected location to achieve a range of different binding activities. Moreover, amino acid residues that are suspected of imparting binding affinity onto the either or both of the binding domains within an ADCC targeting molecule can be preselected, altered and tested in one or more binding assays well known in the art, including functional assays employing ADCC target cell killing. Finally, combinations of the above approaches also can be employed to achieve productive changes in binding affinity of either or both the antigen biding domain or the F_{c} receptor binding domain.

Combinatorial libraries, stochastic libraries and surface expression libraries additionally can be used to make and screen for variants of ADCC targeting molecule species that exhibit altered binding activity or specificity for target antigen or for F_{c} receptors. Such methods enable the rapid construction and screening of large numbers of different ADCC targeting molecule variants. These methods can be directed to one or a few amino acid positions within the ADCC targeting molecule, or alternatively, many positions, including all positions within the antigen or F_{c} receptor binding domains, can be changed and the resultant products screened for the identification of variants having one or more desired binding properties.

For example, a library can be made where particular binding site positions are substituted with all or a subset of the twenty naturally occurring amino acids and then screened for an increase or decrease in binding affinity for antigen or an F_{c} receptor. Similarly, a library of all possible amino acid positions within an antigen or F_{c} receptor binding domain can be substituted with all or a subset of the naturally occurring amino acids and screened to identify a desired change in binding affinity. As described above in reference to mutagenesis of preselected residues, amino acid residues influencing binding characteristics are not restricted to the actual binding domain. Changes in binding characteristics also can be imparted through alterations in amino acid residues outside of the binding regions. Therefore, the methods described above which employ the making and screening of populations of different variants are similarly applicable alone or in combination with the production variants in non-binding domain regions of the ADCC targeting molecule.

Combinatorial, stochastic and surface expression libraries therefore allow for the routine alteration and identification of ADCC targeting molecule that exhibit either enhanced or reduced binding affinity for modified domain. All of such methods are well known to those skilled in the art. For example, combinatorial libraries for generating diverse populations of ADCC targeting molecules can be found described in, for example, Huse et al. Science 240:1275-1281 (1989); Glaser et al. J. of Immunology 149:3903-3913 (1992). Similarly, methods for the construction and screening of a diverse population of binding proteins and variants, including ADCC targeting molecules, can be found described in, for example, of U.S. Pat. Nos. 5,580,717; 5,723,323; and 5,223,409. Methods other than those exemplified above are also well known to those skilled in the art and can additionally be used to make antigen or F_{c} receptor binding variants of ADCC targeting molecules of the invention. Moreover, methods of producing and screening populations of variants to identify desired changes in binding characteristics of ADCC targeting molecules can be used in combination with site-directed mutagenesis methods and other recombinant engineering methods known in the art for producing modifications in a polypeptide.

Screening of variants with preselected changes or large populations produced by combinatorial or stochastic synthesis can be accomplished using a variety of methods well known in the art for determining the binding affinity of a polypeptide or compound. Additionally, methods based on determining the relative binding affinity of a binding molecule to its cognate ligand by comparing the amount of binding between the variant or species within a variant population and the parent ADCC targeting molecule can similarly be used for the identification of variants exhibiting a desired, altered binding affinity. All of such methods are similarly applicable for making and confirming alterations in ADCC targeting molecule type, configuration and valency as well. Such binding methods can be performed, for example, in solution or in solid phase and in a variety of different formats. Binding assays and formats well known in the art and include, for example, immobilization to filters such as nylon or nitrocellulose; two-dimensional arrays, enzyme linked immunosorbant assay (ELISA), radioimmune assay (RIA), panning and plasmon resonance. Such methods can be found described in, for example, the references cited above and in Sambrook et al., supra, and Ansubel et al.

Another method for screening populations or for measuring the affinity of individual altered variable region polypeptides is through surface plasmon resonance (SPR). This method is based on the phenomenon which occurs when surface plasmon waves are excited at a metal/liquid interface. Light is directed at, and reflected from, the side of the surface not in contact with sample, and SPR causes a reduction in the reflected light intensity at a specific combination of angle and wavelength. Biomolecular binding events cause changes in the refractive index at the surface layer, which are detected as changes in the SPR signal. The binding event can be either binding association or disassociation between a receptor-ligand pair. The changes in refractive index can be measured essentially instantaneously and therefore allows for determination of the individual components of an affinity constant. Methods for measuring binding affinity using surface plasmon resonance are well known in the art and can be found described in, for example, Jonsson and Malmquist, Advances in Biosensor, 2:291-336 (1992) and Wu et al. Proc. Natl. Acad. Sci. USA, 95:6037-6042 (1998). Moreover, one apparatus well known in the art for measuring binding interactions is a BIAcore 2000 instrument which is commercially available through Pharmacia Biosensor, (Uppsala, Sweden).

Detection methods for identification of binding species within the population of altered variable regions can be direct or indirect and can include, for example, the measurement of light emission, radioisotopes, calorimetric dyes and fluorochromes. Direct detection includes methods that operate without intermediates or secondary measuring procedures to assess the amount of bound antigen or ligand. Such methods generally employ ligands that are themselves labeled by, for example, radioactive, light emitting or fluorescent moieties. In contrast, indirect detection includes methods that operate through an intermediate or secondary measuring procedure. These methods generally employ molecules that specifically react with the antigen or ligand and can themselves be directly labeled or detected by a secondary reagent.

Using any of the above described screening methods, as well as others well known in the art, an ADCC targeting molecule having altered binding activity toward either or both the target antigen or an F_{c} receptor is identified by comparing the binding of the variant with the parent ADCC targeting molecule. Variants exhibiting increases in binding affinity or specificity toward the target antigen or F_{c} receptor is indicated by greater relative binding compared to the parent ADCC targeting molecule. Whereas variants exhibiting decreases in binding affinity or specificity is indicated by reduced relative binding compared to the parent.

ADCC killing assays are similarly well known to those skilled in the art. Such functional methods can be used both for determining the binding activity of a particular ADCC targeting molecule modification as well as for determining an effective amount to induce ADCC-mediated killing of a target cell or target cell population. Functional ADCC killing methods additionally can be used for determining the specificity, efficiency or efficacy of an ADCC targeting molecule species for a particular target antigen. The latter use is particularly applicable for rapid identification and selection of candidate ADCC targeting molecule species for use in the methods of the invention. However, all of the methods described herein for assessing the binding or ADCC-mediated killing activity of an ADCC targeting molecule species are equally applicable for the initial screening an selection of candidate ADCC targeting molecule species. Therefore, once ADCC targeting molecule species are identified that have selective binding affinity toward a target cell, they can be combined with one or more other ADCC targeting molecule species selective for different target antigens and formulated in an amount sufficient to induce ADCC-mediated killing as described previously.

Functional methods for measuring the ADCC-mediated killing activity of ADCC targeting molecules are well known to those skilled in the art. Such functional methods are reliable predictors of ADCC-mediated killing activity and of the ADCC-mediated killing efficiency and efficacy of two or more ADCC targeting molecule species against target cells in vitro, in situ and in an individual. Therefore, ADCC targeting molecules mediating ADCC killing activity in functional methods and animal models well known in the art are considered therapeutically useful because they correspondingly exhibit target cell destruction activity in vivo.

One particular ADCC target cell killing assay is a chromium-51 (⁵¹Cr) release assay. In this method, ADCC-mediated activity is determined by measuring the lysis of cultured ⁵¹Cr-labeled target cells. Briefly, target cells are labeled with ⁵”Cr and then exposed to effector cells and an ADCC targeting molecule or formulation of ADCC targeting molecule species. The period of exposure or incubation can vary depending on whether quantitative or qualitative results are desired. Generally, the period of exposure is between about 30 minutes and 6 hours, preferably the period is between about 2-5 hours and more preferably the period is about 3-4 hours. ADCC targeting molecule species are contacted with target cells in concentrations ranging generally from about 10 ng/ml to 500 μg/ml, preferably from about 0.1 μg/ml to 100 μg/ml, more preferably from about 0.5 μg/ml to 10 μg/ml. The release of ⁵¹Cr from the target cells is measured as an indicator of cell lysis or cytotoxicity and is compared, for example, to the incubation of target cells alone, target cells with either effector cells or ADCC targeting molecule species alone, and can additionally include comparison with a complete mixture using an ADCC targeting molecule species known to mediate ADCC killing. The total amount of ⁵¹Cr that can be released is measured and ADCC-mediated killing is calculated as the percent killing of target cells observed with ADCC targeting molecule species in the presence of effector cells as compared to target cells incubated alone.

Other ADCC target cell killing assays for measuring the function of an ADCC targeting molecule or the amount sufficient to induce ADCC-mediated killing of a particular formulation include, for example, a target cell proliferation assay. A target cell proliferation assay measures the ability one or more ADCC targeting molecule species in the presence of effector cells to inhibit proliferation of target cells in vitro by ADCC.

Briefly, target cells are collected from an appropriate source, including for example, cell lines and primary cells isolated from an individual or an animal, and suspended in growth medium to achieve a concentration of about 10⁴ viable cells/ml for each sample to be tested. However, the amount and concentration of target cells can be adjusted according to the available of target cells. Effector cells are added to a sample at a concentration of about 10⁶ viable cells/ml, or different ratios of effector to target cells can be can be plated to determine the ADCC-mediated inhibition of target cell proliferation at differing effector cell concentrations. The ratios of effector to target cells can be between about 500:1 to 2:1. ADCC targeting molecule species are added to each sample of target and effector cells and the mixtures are incubated for sufficient time to induce ADCC, generally between about 12-36 hr. Rate of target cell proliferation is determined by measuring the level of DNA synthesis by, for example, ³H-thymidine incorporation, or by measuring the number of cells over time or at the end of the incubation period. Other means of measuring proliferation which can be used are will known in the art. Control samples of effector and target cells without ADCC targeting molecule species are used as a comparison for inhibition of target cell proliferation. Percent inhibition of proliferation as compared to the control samples is calculated as a measure of the ability of the ADCC targeting molecule species to induce ADCC-mediated killing.

Animal models of aberrantly proliferative diseases similarly can be used to assess the binding or ADCC target cell killing activity of an ADCC targeting molecule species or to assess a formulation of ADCC targeting molecule species for an amount sufficient to induce ADCC-mediated killing. Animal models of such pathological conditions well known in the art which are reliable predictors of treatments in human individuals for include, for example, animal models for tumor growth and metastasis, infectious diseases and autoimmune disease.

There are numerous animal tumor models predictive of therapeutic treatment which are well known in the art. These models generally include the inoculation or implantation of a laboratory animal with heterologous tumor cells followed by simultaneous or subsequent administration of a therapeutic treatment. The efficacy of the treatment is determined by measuring the extent of tumor growth or metastasis. Measurement of clinical or physiological indicators can alternatively or additionally be assessed as an indicator of treatment efficacy. Exemplary animal tumor models can be found described in, for example, Brugge et al., Origins of Human Cancer, Cold Spring Harbor Laboratory Press, Plain View, N.Y., (1991).

Similarly, animal models predictive for infectious disease also follow a similar approach. Briefly, laboratory animals are inoculated with an infectious agent and the progression of the infection is monitored by, for example, clinical symptoms, growth culture of the agent from an infected tissue sample or biopsy in the presence or absence of the therapeutic treatment. The reduction in severity of the diagnostic indicator is indicative of the efficacy of the treatment. A variety of animal models for infectious diseases are well known to those skilled in the art.

One animal model predictive for autoimmune diseases is Experimental allergic encephalomyelitis (EAE), also called experimental autoimmune encephalomyelitis. Although originally characterized as a model for neurological autoimmune disease such as human multiple sclerosis, the use of this model to predict treatments of other autoimmune diseases has been widely accepted. EAE is induced in susceptible animals by active immunization with myelin basic protein (MPB) or by passive transfer of MBP-specific T helper lymphocytes. Progression of the disease is characterized by chronic relapsing paralysis and central nervous system demyelination, which can be monitored by observation or by immunological determinants such as delayed-type hypersensitivity (DTH; a measure of cell mediated immunity) response to the immunogen. Efficacy of a therapeutic treatment is compared to progression of the disease in the absence of treatment. A reduction in severity of EAE symptoms or immunological determinants in treated animals is indicative of the efficacy of the therapeutic treatment. For a review of autoimmune disease models see, for example, Urban et al., Cell, 54:577-592 (1988); Brostoff et al., Immunol. Ser. 59:203-218 (1993) and U.S. Pat. Nos. 5,614,192 and 5,612,035.

A growing number of human diseases have been classified as autoimmune and include, for example, rheumatoid arthritis, myasthenia gravis, multiple sclerosis, psoriasis, systemic lupus erythematosus, autoimmune thyroiditis, Graves' disease, inflammatory bowel disease, autoimmune uveoretinitis, polymyositis and diabetes. Animal models for many of these have been developed and can be employed analogously as the EAE model described above predictive assessment of therapeutic treatments for the ADCC-mediated killing methods of the invention. Moreover, treatments ADCC targeting molecule formulations applicable for the treatment of EAE can be reliability extrapolated for the treatment of these and other autoimmune diseases known to those skilled in the art.

Reliable and predictive animal models other than those described above also are well known in the art and similarly can be used to assess various binding and ADCC-mediated functional characteristics of the ADCC targeting molecule species and formulations of the invention. Given the teachings and guidance provided herein, those skilled in the art will know which other animal models can be used for determining binding activity, efficacy or amounts of ADCC targeting molecule species useful in the methods of the invention.

The various methods described previously for assessing or confirming various binding and functional characteristics of ADCC targeting molecules and species can additionally be employed in diagnostic procedures for determining the capacity or efficiency of effector cells isolated from an individual. Knowledge of an individuals inherent ability to mediate ADCC-dependent target cell killing allows for adjustment and fine tuning of an ADCC targeting molecule formulation as well as for independent augmentation of that individual's effector cells to augment or optimize efficacy of the methods of the invention.

Briefly, effector cells can be isolated from peripheral blood, bone marrow or tissue samples from an individual using methods well known in the art and exposed to target cells and an effective amount of an ADCC target molecule in, for example, a ⁵¹Cr release assay. Comparison of the percent cell killing of the test effector cells to normal control effector cells will yield a relative level of ADCC-mediated cell killing activity of the test effector cells. Effector cells can be tested for activity by measuring single aliquot samples or, alternatively, differing amounts of effector cells can be titrated in the presence of target cells and an ADCC targeting molecule to determine a dose response curve. The level of ADCC-mediated activity for a particular dose of test effector cells is compared to results obtained with normal control effector cells at the same dose or doses to obtain a relative level of ADCC-mediated cell killing activity. Any of various formats known in the art, such as multi-sample formats, can be used to assess the ADCC-mediated killing activity of one or more individuals.

As described previously for modulating or optimizing the selection of target antigens and ADCC targeting molecule formulations, the same approach can similarly be used to obtain efficacious ADCC targeting molecule formulations in light of a determination of differing levels of ADCC-mediated activity of an individuals effector cells. For example, amounts of ADCC targeting molecules species can be adjusted to positively or negatively compensate for relative differences in ADCC-mediated killing activity of effector cells isolated from the individual compared to a normal standard. Similarly, the target antigen binding affinity, F_{c} receptor binding affinity, valency or the number of ADCC targeting molecule species also can be adjusted to compensate for, or augment, the inherent activity of an individuals effector cells. Other methods known in the art also can be used as well.

Therefore, the invention provides a method of determining an effective amount of an ADCC targeting molecule formulation. The method consists of: (a) contacting a target cell sample in the presence of effector cells with two or more ADCC targeting molecule species selective for different antigens on the surface of the target cell; (b) measuring the amount of ADCC-mediated target cell killing, and (c) optionally repeating steps (a) and (b) one or more times with a different amount of the two or more ADCC targeting molecule species, or with modified variants thereof, to induce ADCC-mediated killing of the target cells. An effective amount of two or more ADCC targeting molecules will result in target cell killing to achieve between an about 1-50% reduction in target cell number, preferably between an about 50-90% reduction in target cell number, more preferably greater than an about 90% reduction in target cell number. An effective amount also can result in target cell killing to achieve a decreased in the rate of proliferation of the target cells.

Also provided is a method of optimizing an ADCC targeting molecule formulation. The method consists of: (a) contacting a target cell sample in the presence of effector cells with an effective amount of two or more ADCC targeting molecule species selective for different antigens on the surface of the target cell; (b) measuring the amount of ADCC-mediated target cell killing, and (c) repeating steps (a) and (b) one or more times with a different amount of the two or more ADCC targeting molecule species, or with modified variants thereof, to induce a preselected level of ADCC-mediated killing of the target cells. An optimal amount of two or more ADCC targeting molecules will result in a preferred level of target cell killing which can be total eradication of the target cells.

Methods of determining and optimizing an effective amount of ADCC targeting molecule formulation can be combined with a method of determining the ADCC-mediated activity of effector cells. The method of determining effector cell activity consists of: (a) exposing a sample of effector cells to target cells and an ADCC targeting molecule species selective for the target cells under conditions sufficient to induce ADCC-mediated cell killing, (b) measuring the level of ADCC-mediated target cell killing in the sample, and (c) comparing the level of ADCC-mediated target cell killing induced by the sample of effector cells with a level killing induced by a control sample of effector cells, wherein a similar or relative difference in the level of killing is indicative of normal, enhanced or reduced levels of ADCC-mediated target cell killing in the sample of effector cells. The effector cells can be obtained or isolated from an individual.

The invention also provides a method of treating a pathological condition characterized by aberrant cell growth. The method consists of administering an effective amount of two or more ADCC targeting molecule species selective for different antigens expressed on the surface of cells mediating the pathological conditions.

The ADCC-mediated killing methods of the invention for destroying target cells are applicable to a wide variety of pathological conditions. Such applications include those requiring highly selective removal of target cells amongst a heterogenous population of non-target cells as well as those involving less selective obliteration of target cells or of a class of target cells such as a tissue. Additionally, the ADCC-mediated killing methods of the invention also can be used to simultaneously target multiple different target cells or tissues for the treatment of one or more pathological conditions. Modulation of the target cell selectivity, including activity and efficacy, will depend on the pathological condition and the extent to which elimination targets cells are desired for treatment or reduction in the severity of the pathological condition. Modulation can be accomplished by adjusting the ADCC targeting molecule species, formulation, type, configuration, antigen binding activity and/or F_{c} receptor binding activity as described previously.

In one embodiment, application of the methods of the invention recognize that there are two broad categories of target cells or tissues that can be targeted for elimination in treating or reducing the severity of a pathological condition. One category consists of pathological conditions derived from, or associated with, cells or tissues that are non-essential for survival or maintenance of the quality of life of an individual. Exemplary of such tissues include, for example, the prostate and breast. In the other category are pathological conditions derived from or associated with cells or tissues that perform essential functions for the survival or maintenance of the quality of life of an individual. Cells and tissues performing essential functions include, for example, the brain, liver, lung and hematopoietic system.

Recognition of these categories of target cells and tissues allows for further modulation of the efficacy of the ADCC-mediated killing methods of the invention. In this regard, ADCC targeting molecules selective for the aberrant target cells within a heterogenous population of non-target cells is unnecessary if the pathological condition is associated with a non-essential tissue. For example, in prostate or breast cancer, it is sufficient to target prostate or breast cells of those tissues to treat or reduce the severity of the pathological condition. Targeting of all the cells within these tissues will result in the killing of both normal and cancerous cells. Although such a treatment will obliterate the tissue, it also will necessarily eradicate the aberrant cancerous cells. As the tissue is non-essential for the survival and quality of life, removal will therefore improve the health of the individual. Therefore, in treating pathological conditions of non-essential tissues it is sufficient that the ADCC targeting molecules bind the aberrant target cells and are at least selective for the non-essential tissue from which the target cells are associated with or from which they derive. Expanding the scope of ADCC targeting molecule specificity to the non-essential tissue compared to the aberrant target cells allows for a wider range of selection of target antigens and formulations of ADCC targeting molecules.

The same methods for treating a pathological condition are employed whether the target cells associated with the pathological condition are within essential or non-essential tissue. In this regard, the ADCC-mediated killing methods described previously, which utilize two or more ADCC targeting molecule species selective for different antigens on the surface of target cells, are similarly employed to eradicate cells of the pathological condition that are characterized by aberrant cell growth. The target cells will be, however, those cells mediating the pathological condition or those cells exhibiting deleterious pathological effects. Such cells are characterized by aberrant regulation or cell growth, for example. Contacting these cells, by administration to the individual, with two or more ADCC targeting molecule species selective for different surface antigens will result in selective ADCC-mediated killing of these cells with concomitant treatment or reduction in the severity of the disease.

As described previously in regard to a method of inducing ADCC against a target cell, an effective amount of ADCC targeting molecule species when used to treat an pathological condition is similarly an amount of two or more ADCC targeting molecule species required to effect a decrease in the amount of target cell number or rate of proliferation when administered to an individual. The dosage of an ADCC targeting molecule formulation required to be therapeutically effective will depend, for example, on the pathological condition characterized by aberrant cell growth to be treated, the route and form of administration, the weight and condition of the individual, and previous or concurrent therapies. The appropriate amount considered to be an effective dose for a particular application of the method can be determined by those skilled in the art, using the guidance provided herein. For example, the amount can be extrapolated from in vitro or in vivo assays as described previously. One skilled in the art will recognize that the condition of the patient can be monitored throughout the course of therapy and that the amount of the ADCC targeting molecule formulation that is administered can be adjusted accordingly.

For treating or reducing the severity of a pathological condition, an effective amount is an efficious amount of two or more ADCC targeting molecule species selective for different antigens on the surface of target cells associated with the pathological condition. An effective amount can be, for example, between about 10 μg/kg to 500 mg/kg body weight, for example, between about 0.1 mg/kg to 100 mg/kg, or preferably between about 1 mg/kg to 50 mg/kg, depending on the treatment regimen. For example, if an ADCC targeting molecule formulation is administered from one to several times a day, then a lower dose would be needed than if a formulation were administered weekly, or monthly or less frequently. Similarly, formulations that allow for timed-release of an ADCC targeting molecule formulation would provide for the continuous release of a smaller amount of ADCC targeting molecule species than would be administered as a single bolus dose. For example, an ADCC targeting molecule formulation can be administered at between about 1-5 mg/kg/week.

A formulation of two or more ADCC targeting molecule species can be delivered systemically, such as intravenously or intra arterially. An ADCC targeting molecule formulation also can be administered locally at a site of the pathological condition. Appropriate sites for administration of an ADCC targeting molecule formulation are known, or can be determined, by those skilled in the art depending on the clinical indications of the individual being treated. For example, an ADCC targeting molecule formulation, capable of inducing ADCC-mediated killing of target cells can be provided as isolated and substantially purified polypeptides in pharmaceutically acceptable formulations using formulation methods known to those of ordinary skill in the art. These formulations can be administered by standard routes, including for example, topical, transdermal, intraperitoneal, intracranial, intracerebroventricular, intracerebral, intravaginal, intrauterine, oral, rectal or parenteral (e.g., intravenous, intraspinal, subcutaneous or intramuscular) routes. Methods for such routes of administration are well known to those skilled in the art.

In addition, an ADCC targeting molecule formulation can be incorporated into biodegradable polymers allowing for sustained release of the compound, the polymers being implanted in the vicinity of where drug delivery is desired, for example, at the site of a tumor or implanted so that the ADCC targeting molecule species are released systemically over time. Osmotic minipumps also can be used to provide controlled delivery of specific concentrations of ADCC targeting molecule species and formulations through cannulae to the site of interest, such as directly into a tumor growth or into the vascular supply a tumor. The biodegradable polymers and their use are described, for example, in detail in Brem et al., J. Neurosurg. 74:441-446 (1991).

The invention provides compositions of two or more ADCC targeting molecule species and variants thereof, together with a pharmaceutically acceptable medium and formulations. Such compositions can be used in the ADCC-mediated killing methods of the invention to inhibit treat or reduce the severity of a pathological condition. For example, an ADCC targeting molecule formulation containing two or more ADCC targeting molecule species, or variants thereof, can be administered as a solution or suspension together with a pharmaceutically acceptable medium. Such a pharmaceutically acceptable medium can be a human pharmaceutical grade, sterile medium, such as, for example, water, sodium phosphate buffer, phosphate buffered saline, normal saline or Ringer's solution or other physiologically buffered saline, or other solvent or vehicle such as a glycol, glycerol, an oil such as olive oil or an injectable organic ester. Pharmaceutically acceptable media can be substantially free from contaminating particles and organisms. The terms “pharmaceutically acceptable medium” and “pharmaceutically acceptable formulation” are intended to mean that the mediums admixed with ADCC targeting molecule species are of sufficient purity and quality for use in humans.

The ADCC targeting molecule formulations include those applicable for parenteral administration such as subcutaneous, intraperitoneal, intramuscular, intravenous, intradermal, intracranial, intratracheal, and epidural administration. As well as formulations applicable for oral, rectal, ophthalmic (including intravitreal or intracameral), nasal, topical (including buccal and sublingual), intrauterine, or vaginal administration. The ADCC targeting molecule formulations or variants thereof, can be presented in unit dosage form and can be prepared by pharmaceutical techniques well known to those skilled in the art. Such techniques include the step of bringing into association the active ingredient and a pharmaceutical carrier or excipient.

Formulations suitable for parenteral administration include aqueous and non-aqueous sterile injection solutions such as the pharmaceutically acceptable mediums described above. The solutions can additionally contain, for example, anti-oxidants, buffers, bacteriostats and solutes which render the formulation isotonic with the blood of the intended recipient. Other formulations include, for example, aqueous and non-aqueous sterile suspensions which can include suspending agents and thickening agents. The formulations can be presented in unit-dose or multi-dose containers, for example, sealed ampules and vials, and can be stored in a lyophilized condition requiring, for example, the addition of the sterile liquid carrier, immediately prior to use. Extemporaneous injection solutions and suspensions can be prepared from sterile powders, granules and tablets of the kind previously described.

A pharmaceutically acceptable medium can additionally contain physiologically acceptable compounds that act, for example, to stabilize the ADCC targeting molecule species. Such physiologically acceptable compounds include, for example, carbohydrates such as glucose, sucrose or dextrans; antioxidants such as ascorbic acid or glutathione; chelating agents such as EDTA, which disrupts microbial membranes; divalent metal ions such as calcium or magnesium; low molecular weight proteins; lipids or liposomes; or other stabilizers or excipients. As described previously, an ADCC targeting molecule formulation also can be formulated with a pharmaceutically acceptable medium such as a biodegradable polymer.

Formulations of two or more ADCC targeting molecule species, variants and combinations thereof can also be delivered in an alternating administrations so as to combine their ADCC target cell killing effects over time. For example, an ADCC targeting molecule formulation containing two or more ADCC targeting molecule species can be administered in a single bolus dose followed by multiple administrations of one or more ADCC targeting molecule species or variant alone, or in combination with a different ADCC targeting molecule formulation. Whether simultaneous or alternating delivery of the ADCC targeting molecule formulation, variant or combination thereof, the mode of administration can be any of those types of administrations described previously and will depend on the particular therapeutic need and efficacy of the ADCC targeting molecule formulation selected for the purpose. Determining which ADCC targeting molecule formulations, species and variants to combine in a temporally administered regime, will depend on the pathological condition to be treated and the specific physical characteristics of the individual affected with the disease. Those skilled in the art will know or can determine a specific regime of administration which is effective for a particular application using the teachings and guidance provided herein together with diagnostic and clinical criteria known within the field of art of the particular pathological condition.

The methods of treating a pathological condition characterized by aberrant cell growth additionally can be practiced in conjunction with other therapies. For example, for treating cancer, the methods of the invention can be practiced prior to, during, or subsequent to conventional cancer treatments such as surgery, chemotherapy, including administration of cytokines and growth factors, radiation or other methods known in the art. Similarly, for treating pathological conditions which include infectious disease, the methods of the invention can be practiced prior to, during, or subsequent to conventional treatments, such as antibiotic administration, against infectious agents or other methods known in the art. Treatment of pathological conditions of autoimmune disorders also can be accomplished by combining the ADCC-mediated killing methods of the invention with conventional treatments for the particular autoimmune diseases. Conventional treatments include, for example, chemotherapy, steroid therapy, insulin and other growth factor and cytokine therapy, passive immunity, inhibitors of T cell receptor binding and T cell receptor vaccination. The methods of the invention can be administered in conjunction with these or other methods known in the art and at various times prior, during or subsequent to initiation of conventional treatments. For a description of treatments for pathological conditions characterized by aberrant cell growth see, for example, The Merck Manual, Sixteenth Ed, (Berkow, R., Editor) Rahway, N.J., 1992.

As described above, administration of an ADCC targeting molecule formulation can be, for example, simultaneous with or delivered in alternative administrations with the conventional therapy, including multiple administrations. Simultaneous administration can be, for example, together in the same formulation or in different formulations delivered at about the same time or immediately in sequence. Alternating administrations can be, for example, delivering an ADCC targeting molecule formulation and a conventional therapeutic treatment in temporally separate administrations. As described previously, the temporally separate administrations of an ADCC targeting molecule formulation and conventional therapy can similarly use different modes of delivery and routes.

Additionally, the methods of treating a pathological condition characterized by aberrant cell growth using two or more ADCC targeting molecule species also can be performed in conjunction with various cell therapies. One cell therapy is co-administration of effector cells to augment the capacity, efficiency or efficacy of the administered ADCC targeting molecule species. Effector cells can be obtained or isolated and expanded in culture to generate a source of effector cells for administration. Administration of additional effector cells will supplement the capacity of total effector cell activity in the recipient. Alternatively, the effector cells can be genetically modified, for example, to increase the expression of stimulatory F_{c} receptors or to decrease the expression of inhibitory F_{c} receptors. Administration of such F_{c} receptor-modified effector cells will increase both the capacity and efficiency of ADCC-mediated killing in the recipient individual. These effector cell therapy methods alone or combined with other methods known in the art for augmenting effector cell activity can be co-administered to an individual being treated with two or more ADCC targeting molecules selective for the target cell increase treatment efficacy against the pathological condition.

Effector cells for co-administration can be autologous or allogeneic effector cells. Therefore, the effector cells can be obtained, for example, from the individual afflicted with the pathological condition or from an individual that is substantially histocompatible to the recipient individual. Methods for the isolation, purification or fractionation of effector cells are well known in the art. Similarly, methods for typing histocompatiblity antigens to determine immunological compatibility also are well known in the art. Using such antigen typing methods, those skilled in the art will know or can determine what level of antigen similarity is necessary for an effector cell type to be immunologically compatible with a recipient individual. The tolerable differences between a donor cell and a recipient can vary with different tissues and are known or can be readily determined by those skilled in the art. Methods for assessing transplantation antigen compatibility are well known to those skilled in the art.

Moreover, additional methods well known in the art can be used to reduce the severity of an anti-graft immune response to further increase the in vivo viability of allogeneic effector cells. Therefore, for therapeutic applications, it is not necessary to select autologous effector cells from the individual exhibiting or predisposed to a pathological condition amenable to treatment using the methods of the invention. Instead, alternative methods can be employed which can be used in conjunction with allogeneic effector cells to confer sufficient viability onto the co-administered cells to augment ADCC-mediated killing. Such additional methods include, for example, immunosuppressive agents, which can be used to render the host immune system tolerable to engraftment of the co-administered effector cells. The regimen and type of immunosuppressive agent to be administered will depend on the degree of MHC similarity between the effector cell and the recipient. Those skilled in the art know, or can determine, what level of histocompatiblity between donor and recipient antigens is applicable for use with one or more immunosuppressive agents. Following standard clinical protocols, administration and dosing of such immunosuppressive agents can be adjusted to improve efficiency of engraftment and the viability of the co-administered effector cells. Specific examples of immunosuppressive agents useful for reducing a host anti-graft immune response include, for example, cyclosporin, corticosteroids, and the immunosuppressive antibody known in the art as OKT3.

The type of effector cell chosen will depend, for example, on the need for augmentation and the pathological condition. If ADCC-mediated killing is to be increased or even maximized, it can be desirable to select those effector cells who exhibit ADCC functions as a biological role in innate immunity of an individual. Alternatively, if cell-type characteristics are desired to treat a specific type of pathological condition, it can be desirable to take cells of that cell type and modify them to enhance their effector cell function. Those skilled in the art will know which effector cells are suitable for use in conjunction with the ADCC-mediated methods of the invention.

The temporal order of administration of the effector cells is unimportant so long as the cells become available for recognition of the ADCC targeting molecule species and destruction of the target cells. Therefor, co-administration can be accomplished by, for example, simultaneous administration as well as by prior or post administration of the ADCC targeting molecules to the individual. Administration can be achieved using well known methods in the art such as by injection into the circulatory system, subcutaneous implantation or surgical implantation or graftment.

Similarly, other cell therapy methods well know in the art can additionally be employed in conjunction with the ADCC-mediated killing methods of the invention. Such other methods include, for example, cell replacement therapy for the regeneration or reconstitution of tissues and cellular components thereof, and cell therapy using genetically modified cells for the production of a therapeutic protein or macromolecule. Specific examples of cell replacement therapy include hematopoietic stem and progenitor cell therapy, which can be used, for example, to reconstitute ablated bone marrow cells of cancer patients, and neuronal stem and progenitor cell therapy for the treatment of Parkinson's disease. Cell therapy for production of a therapeutic protein includes, for example, the transplantation of a variety of cell types and progenitors thereof genetically modified to produce, for example, insulin, other cytokines, growth factors, and enzymes. Such genetic cell therapies are applicable, for example, to the treatment of diabetes, cancer. Various other examples of cell replacement and genetic cell therapy are well known in the art and are similarly applicable for use in conjunction with the methods of the ADCC-mediated killing methods of the invention.

The invention further provides a method of purging bone marrow cells of pathogenic cells. The method consists of contacting bone marrow cells in the presence of effector cells with an effective amount of two or more ADCC targeting molecules selective for different antigens on the pathogenic cells. The method is applicable for the in vivo and ex vivo treatment of hematopoietic diseases.

Any and all of the methods described previously for inducing ADCC-mediated killing of a target cell and for treating a pathological condition characterized by aberrant cell growth also can be used to purge bone marrow cells of unwanted cellular constituents. Such unwanted cellular constituents can include, for example, cancer cells, autoimmune cells pathogenic agents and cells infected with pathogenic agents. The bone marrow cells can be removed and treated with two or more ADCC targeting molecule species selective for different antigens on the unwanted target cells as described previously to obtain a resultant population of bone marrow cells substantially depleted of the target cells. The purged population can be re-administered to the individual or expanded in culture and re-administration either with or without prior bone marrow ablation of that individual. Methods for the removal, expansion and ablation of bone marrow cells are well known in the art.

Moreover, any and all of the method described previously for inducing ADCC-mediated killing of a target cell and for treating a pathological condition characterized by aberrant cell growth additionally can be used in vitro to isolate a particular cell type. For example, for cell therapy methods it can be desirable to employ stem or progenitor cells in the cell replacement. The methods of ADCC-mediated target cell killing of the invention can therefore be used to isolate substantially pure populations of such cells in vitro for subsequent therapeutic applications. Additionally, such methods as applied to the isolation of particular cell types is not restricted to cell types amenable to therapeutic applications. Instead, the methods of the invention can be used to obtain a substantially pure population of a particular cell type for any particular purpose, including for example, scientific research and diagnostic applications. To obtain a substantially pure cell population consisting of a particular cell type or cell types, the method employs the use of two or more ADCC targeting molecule species for those undesired cell types within the initial sample. In the presence of effector cells, the undesired target cells will be destroyed, leaving the desired cell type remaining. The effector cells can be removed, or alternatively engineered with a suicide gene which can be activated to eliminated effector cells from the resultant population. Given the teachings and guidance provided herein, those skilled in the art will known how to apply the methods of the invention for isolation of a substantially pure population of cells, including stem and progenitor cells.

TABLE 1

| | |
|---|---|
| HEMATOPORETIC NEOPLASMS | Chronic Myclogenous |
| Lymphoid Neoplasms | Leukemia (CML) |
| Myeloid Neoplasms | Polycythemia Vera |
| Histiocytoses | Essential Thrombocytosis |
| Precursor B lymphoblastic | Myelofibrosis with Myeloid |
| leukemia/lymphoma (ALL) | Metaplasia |
| Precursor T lymphoblastic | Hemangioma |
| leukemia/lymphoma (ALL) | Lymphangioma |
| Chronic lymphocytic | Glomangioma |
| leukemia/small lymphocytic | Kaposi Sarcoma |
| lymphoma (SLL) | Hemanioendothelioma |
| Lymphoplasmacytic lymphoma | Angiosarcoma |
| Mantle cell lymphoma | Hemangiopericytoma |
| Follicular lymphoma | HEAD & NECK |
| Marginal zone lymphoma | Basal Cell Carcinoma |
| Hairy cell leukemia | Squamous Cell Carcinoma |
| Plasmacytoma/plasma cell | Ceruminoma |
| myeloma | Osteoma |
| Diffuse large B-cell | Nonchromaffin Paraganglioma |
| lymphoma | Acoustic Neurinoma |
| Burkitt lymphoma | Adenoid Cystic Carcinoma |
| T-cell chronic lymphocytic | Mucoepidermoid Carcinoma |
| leukemia | Malignant Mixed Tumors |
| Large granular lymphocytic | Adenocarcinoma |
| leukemia | Lymphoma |
| Mycosis fungoids and sezary | Fibrosarcoma |
| syndrome | Osteosarcoma |
| Peripheral T-cell lymphoma, | Chondrosarcoma |
| unspecified | Melanoma |
| Angioimmunoblastic T-cell | Olfactory Neuroblastoma |
| lymphoma | Isolated Plasmocytoma |
| Angiocentric lymphoma | Inverted Papillomas |
| (NK/T-cell lymphoma) | Undifferentiated Carcinoma |
| Intestinal T-cell lymphoma | Mucoepidermoid Carcinoma |
| Adult T-cell | Acinic Cell Carcinoma |
| leukemia/lymphoma | Malignant Mixed Tumor |
| Anaplastic large cell | Other Carcinomas |
| lymphoma | Amenoblastoma |
| Hodgkin Diseases (HD) | Odontoma |
| Acute myclogenous leukemia | THYMUS |
| (AML) | Malignant Thymoma |
| Myclodysplastic syndromes | Type I (Invasive thymoma) |
| Chronic Myclofroliferative | Carcinoma of the |
| Disorders | Gallbladder |
| Type II (Thymic carcinoma) | Adenocarcinoma |
| Squamous cell carcinoma | Squamous Cell Carcinoma |
| Lymph epithelioma | Papillary, poorly |
| THE LUNG | differentiated |
| Squamous Cell Carcinoma | THE PANCREAS |
| Adenocarcinoma | Adenocarcinoma |
| Bronchial derived | Cystadenocarcinoma |
| Acinar; papillary; solid | Insulinoma |
| Bronchioalveolar | Gastrinoma |
| Small Cell Carcinoma | Glucagonamoa |
| Oat Cell | THE KIDNEY |
| Intermediate Cell | Renal Cell Carcinoma |
| Large Cell Carcinoma | Nephroblastoma (Wilm's |
| Undifferentiated; giant | Tumor) |
| cell; clear cell | THE LOWER URINARY TRACT |
| Malignant Mesothelioma | Urothelial Tumors |
| Sarcomotoid Type | Squamous Cell Carcinoma |
| Epithelial Type | Mixed Carcinoma |
| THE GASTROINTESTINAL | Adenocarcinoma |
| TRACT | Small Cell Carcinoma |
| Squamous Cell Carcinoma | Sarcoma |
| Adenocarcinoma | THE MALE GENITAL TRACT |
| Carcinoid | Squamous Cell |
| Malignant Melanoma | CarcinomaSarcinoma |
| Adenocarcinoma | Speretocytic Sarcinoma |
| Gastric Carcinoma | Embyonal Carcinoma |
| Gastric Lymphoma | Choriocarcinoma |
| Gastric Stromal Cell Tumors | Teratoma |
| Lymphoma | Leydig Cell Tumor |
| Kaposi's Sarcoma | Sertoli Cell Tumor |
| Intestinal Stromal Cell | Lymphoma |
| Tumors | Adenocarcinoma |
| Carcinids | Undifferentiated Prostatic |
| Malignant Mesethelioma | Carcinoma |
| Non-mucin producing | Ductal Transitional |
| adenocarcinoma | Carcinoma |
| THE LIVER AND THE BILIARY | THE FEMALE GENITAL TRACT |
| TRACT | Squamous Cell Carcinoma |
| Hepatocellular Carcinoma | Basal Cell Carcinoma |
| Cholangiocarcinoma | Anoplastic Carcinoma |
| Hepatoblastoma | Adenoma |
| Angiosarcoma | Carcinoma |
| Fibrolameller Carcinoma | Pheochromocytoma |
| Melanoma | Neuroblastome |
| Fibrosarcoma | Paraganglioma |
| Intaepithelial Carcinoma | Pineal |
| Adenocarcinoma Embryonal | Pineoblastoma |
| Rhabdomysarcoma | Pineocytoma |
| Large Cell Carcinoma | THE SKIN |
| Neuroendocrine or Oat Cell | Melanoma |
| Carcinoma | Squamous cell carcinoma |
| Adenocarcinoma | Basal cell carcinoma |
| Adenosquamous Carcinoma | Merkel cell carcinoma |
| Undifferentiated Carcinoma | Extramarnary Paget's Disease |
| Carcinoma | Paget's Disease of the |
| Adenoacanthoma | nipple |
| Sarcoma | Kaposi's Sarcoma |
| Carcinosarcoma | Cutaneous T-cell lymphoma |
| Leiomyosarcoma | BONES, JOINTS, AND SOFT |
| Endometrial Stromal Sarcoma | TISSUE TUMORS |
| Serous Cystadenocarcinoma | Multiple Myeloma |
| Mucinous Cystadenocarcinoma | Malignant Lymphoma |
| Endometrioid Tumors | Chondrosacrcoma |
| Adenosarcorna | Mesenchymal Chondrosarcoma |
| Celioblastoma (Brenner | Osteosarcoma |
| Tumor) | Ewing Tumor (Ewing Sarcoma) |
| Clear Cell Carcinoma | Malignant Giant Cell Tumor |
| Unclassified Carcinoma | Adamantinoma |
| Granulosa-Theca Cell Tumor | Malignant Fibrous |
| Sertoli-Leydig Cell Tumor | Histiocytoma |
| Disgerminoma | Desmoplastc Fibroma |
| Teratoma | Fibrosarcoma |
| THE BREAST | Chordoma |
| Phyllodes Tumor | Hemangioendothelioma |
| Sarcoma | Memangispericytoma |
| Paget's Disease | Liposarcoma |
| Carcinoma | Malignant Fibrous |
| Insitu Carcinoma | Histiocytoma |
| Invasive Carcinoma | Rhabdomysarcoms |
| THE ENDOCRINE SYSTEM | Leiomyosarcoma |
| Adenoma | Angiosarcoma |
| Carcinoma | |
| Meningnoma | |
| Cramiopharlingioma | |
| Papillary Carcinoma | |
| Follicular Carcinoma | |
| Medullary Carcinoma | |
| NERVOUS SYSTEM | |
| Schwannoma | |
| Neurofibroma | |
| Malignant Periferal Nerve | |
| Sheath Tumor | |
| Astrocytoma | |
| Fibrillary Astrocytoma | |
| Glioblastoma Multiforme | |
| Brain Stem Glioma | |
| Pilocytic Astrocytoma | |
| Pleomorphic | |
| Xanthorstrocytoma | |
| Oligodendroglioma | |
| Ependymoma | |
| Gangliocytoma | |
| Cerebral Neuroblastoma | |
| Central Neurocytoma | |
| Dysembryoplastic | |
| Neuroepithelial Tumor | |
| Medulloblastoma | |
| Malignant Meningioma | |
| Primary Brain Lymphoma | |
| Primary Brain Germ Cell | |
| Tumor | |
| THE EYE | |
| Carcinoma | |
| Squamous Cell Carcinoma | |
| Mucoepidermoid Carcinoma | |
| Melanoma | |
| Retinoblastoma | |
| Glioma | |
| Meningioma | |
| THE HEART | |
| Myxoma | |
| Fibroma | |
| Lipoma | |
| Papillary Fibroelastoma | |
| Rhasdoyoma | |
| Angiosarcoma | |
| Other Sarcoma | |
| HISTIOCYTOSES | |
| Langerhans Cell | |
| Histiocytosis | |

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific details are only illustrative of the invention. It is understood that modifications which do not substantially affect the activity of the various embodiments of this invention are also included within the definition of the invention provided herein. Therefore, it should be understood that various modifications can be made without departing from the spirit of the invention.

What is claimed is:

1. A composition, comprising two or more ADCC targeting molecules each selective for different antigens on the surface of a target cell and in a pharmaceutically acceptable medium.

2. The composition of claim 1, wherein said two or more ADCC targeting molecules further comprise more than two binding species.

3. The composition of claim 1, wherein said two or more ADCC targeting molecules further comprise subunits of a pentameric binding molecule.

4. The composition of claim 1, wherein said two or more ADCC targeting molecules further comprise an antibody or functional variant thereof.

5. A composition, comprising three or more ADCC targeting molecules.

6. The composition of claim 5, wherein said three or more ADCC targeting molecules further comprise more than three binding species.

7. The composition of claim 5, wherein said three or more ADCC targeting molecules further comprise subunits of a pentameric binding molecule.

8. The composition of claim 5, wherein said three or more ADCC targeting molecules further comprises an antibody or functional variant thereof.

9. The composition of claim 5, further comprising a pharmaceutically acceptable medium.

10. A composition, comprising effector cells and two or more ADCC targeting molecule species in a pharmaceutically acceptable medium.

11. The composition of claims 1, 5 or 10, further comprising an F_{c} receptor stimulatory compound.

12. The composition of claim 11, wherein said stimulatory compound is γ-interferon or an adjuvant.

13. A method of inducing antibody-dependent cell cytotoxicity (ADCC) against a target cell comprising contacting said target cell in the presence of effector cells with two or more ADCC targeting molecule species each selective for different antigens on the surface of said target cell.

14. The method of claim 13, wherein said two or more ADCC targeting molecules further comprise three or more species.

15. The method of claim 13, wherein said two or more ADCC targeting molecules further comprise subunits of a pentameric binding molecule.

16. The method of claim 13, wherein said ADCC targeting molecule further comprises an antibody or functional variant thereof.

17. The method of claim 13, wherein at least one of said different antigens is a cellular antigen.

18. The method of claim 13, wherein at least one of said different antigens is a heterologous antigen.

19. The method of claim 13, wherein at least one of said different antigens is present at abundant levels on the surface of said target cell.

20. The method of claim 13, wherein at least one of said different antigens is present at moderate levels on the surface of said target cell.

21. The method of claim 13, wherein at least one of said different antigens is present at low levels on the surface of said target cell.

22. The method of claim 13, wherein said target cell further comprises a cancer cell.

23. The method of claim 13, wherein said target cell is selected from the group consisting of prostate carcinoma, breast, lung and skin cancer.

24. The method of claim 13, wherein said target cell contains an infectious agent.

25. The method of claim 13, wherein said ADCC effector cells further comprise NK cells, macrophages, monocytes, neutrophils or eosinophils.

26. A method of treating a pathological condition characterized by aberrant cell growth comprising administering an effective amount of two or more ADCC targeting molecules selective for different antigens expressed on the surface of cells mediating the pathological condition.

27. The method of claim 26, wherein said two or more ADCC targeting molecules further comprise three or more species.

28. The method of claim 26, wherein said two or more ADCC targeting molecules further comprise subunits of a pentameric binding molecule.

29. The method of claim 26, wherein said ADCC targeting molecule further comprises an antibody or functional variant thereof.

30. The method of claim 26, wherein at least one of said different antigens is a cellular antigen.

31. The method of claim 26, wherein at least one of said different antigens is a heterologous antigen.

32. The method of claim 26, wherein at least one of said different antigens is present at abundant levels on the surface of said target cell.

33. The method of claim 26, wherein at least one of said different antigens is present at moderate levels on the surface of said target cell.

34. The method of claim 26, wherein at least one of said different antigens is present at low levels on the surface of said target cell.

35. The method of claim 26, wherein said pathological condition further comprises cancer.

36. The method of claim 26, wherein said pathological condition is selected from the group consisting of prostate, breast, lung and skin cancer.

37. The method of claim 26, wherein said pathological condition further comprises an infectious disease.

38. The method of claim 26, wherein said pathological condition further comprises an autoimmune disease.

39. A method of purging bone marrow cells of pathogenic cells comprising contacting bone marrow cells in the presence of effector cells with an effective amount of two or more ADCC targeting molecules selective for different antigens on the pathogenic cells.

40. The method of claim 39, wherein said two or more ADCC targeting molecules further comprise three or more species.

41. The method of claim 39, wherein said two or more ADCC targeting molecules further comprise subunits of a pentameric binding molecule.

42. The method of claim 39, wherein said ADCC targeting molecule further comprises an antibody or functional variant thereof.

43. The method of claim 39, wherein at least one of said different antigens is a cellular antigen.

44. The method of claim 39, wherein at least one of said different antigens is a heterologous antigen.

45. The method of claim 39, wherein one of said different antigens is present at abundant levels on the surface of said target cell.

46. The method of claim 39, wherein one of said different antigens is present at moderate levels on the surface of said target cell.

47. The method of claim 39, wherein one of said different antigens is present at low levels on the surface of said target cell.

48. The method of claim 39, wherein said pathogenic cells further comprise cancer cells.

49. The method of claim 39, wherein said pathogenic cells are selected from the group consisting of prostate, breast, lung and skin cancer.

50. The method of claim 39, wherein said pathogenic cells further comprise an infectious agent.

51. The method of claim 39, wherein said pathogenic cells further comprise autoimmune cells.

52. The method of claim 39, wherein said contacting occurs in vivo.

53. The method of claim 39, wherein said contacting occurs ex vivo.
